(19) United States
(12) Patent Application Publication
(10) Pub. No.: US 2026/0268432 A1
(43) Pub. Date: Sep. 10, 2026

(54) DATABASE ACCESS SYSTEM USING MACHINE LEARNING-BASED RELATIONSHIP ASSOCIATION

(71) Applicant: C3S, Inc., Jacksonville Beach, FL (US)

(72) Inventor: Mintu BACHANN, Jacksonville Beach, FL (US)

(73) Assignee: C3S, Inc., Jacksonville Beach, FL (US)

(21) Appl. No.: 19/548,873

(22) Filed: Feb. 24, 2026

Related U.S. Application Data
(63) Continuation-in-part of application No. 17/239,378, filed on Apr. 23, 2021, now Pat. No. 12,681,959.

(51) Int. Cl. G06Q 50/26; G16H 10/20

(52) U.S. Cl. CPC G06Q 50/265; G16H 10/20

(57) ABSTRACT

A system for assessing user need based on relationship association and vulnerability tiering is provided. Some embodiments involve transmitting a questionnaire comprising questions that represent a plurality of categories; receiving responses to the questionnaire; retrieving supplemental user information from one or more data sources; converting the responses and the supplemental user information; assigning, using a machine learning model, weights to the responses based on correlations among the responses; computing base scores for the plurality of categories from the weighted responses using a hybrid scoring system; quantifying, using a combination function, compounding vulnerabilities across categories; applying the compounding vulnerabilities to the base scores to produce a vulnerability index score; comparing the vulnerability index score to thresholds associated with vulnerability tiers to establish a vulnerability tier of a user; and generating and transmitting a vulnerability tier record to the user, including an explanation of the vulnerability tier of the user.

### TECHNICAL FIELD

The present disclosure generally relates to the field of customer service platforms. More specifically, and without limitation, this disclosure relates to systems and methods for database access using machine learning-based relationship association to generate service provider information.

### BACKGROUND

There is a need in the social services industry to improve upon community access to health, education, and social programs. The need for such access is especially important for low-income and other “high risk” groups who may not have access to healthcare and educational resources. Despite the need, current community outreach and social servicing platforms suffer from various drawbacks, such as service fragmentation and geographic isolation, which prevent them from fulfilling the demand. These platforms fail to accurately determine their customers' needs because they provide superficial and inadequate assessment analysis of needs and have limited access to customer information.

Furthermore, existing platforms fall short in their ability to reliably match, synchronize, and track communications between community members and service providers, resulting in fragmented interactions and missed opportunities for timely support. These systems lack the mechanisms needed to support seamless cross-platform communication or integration with external service networks. As a result, users must navigate multiple disconnected systems, which diminishes efficiency and creates barriers to receiving assistance. In addition, existing community outreach and social service platforms exhibit significant security vulnerabilities that expose users' personal information to potential risks. These deficiencies can undermine user trust and raise serious concerns regarding data protection, privacy, and compliance with modern security standards.

In some cases, existing platforms fall short in adequately assessing their users' needs and vulnerabilities because they attempt to identify their users' needs and vulnerabilities with minimally effective questionnaires. These questionnaires are redundant and static, and as a result, do not accurately capture relationships between user responses and/or compounding vulnerabilities. These pitfalls lead to inaccurate assessments matches with service providers, ultimately creating the risk of the platforms' users failing to receive the service they need.

### SUMMARY

The present disclosure provides methods and systems for efficiently determining customer needs using machine learning-based relationship associations to access databases and generate service provider recommendations.

A first aspect of the disclosure provides a system for using machine learning-based relationship association to assess a user need, the system may include at least one processor; and a storage medium storing instructions that, when executed, configure the at least one processor to perform operations. The operations may include receiving from a user device one of an electronic service request or registration request; transmitting to the user device for display on a user interface, over a communication network, a questionnaire with at least a first and second question in response to the request of the user; receiving responses to the first and second questions from the user device over the network; determining, using a machine-learning algorithm, a relationship between the first and second questions based on the received responses; assigning to the first and second questions, using the machine-learning algorithm, a relationship value between 0 and 1 based on the determined relationship, wherein the relationship value represents a likelihood the response to the first question is associated with the response to the second question; determining, using the machine-learning algorithm, whether the first or second question has a relationship value greater than 0.5; designating, using the machine-learning algorithm, the first or second question as a critical question when the relationship value is greater than 0.5; determining, using the machine-learning algorithm, a need of the user based on the response of the user to the critical question; accessing a database to generate a list of service providers based on the determined need of the user; and providing the list of service providers to the user device for display on the user interface.

Another aspect of the disclosure provides a computer-implemented method for generating a list including at least one service provider. The method may include receiving from a user on a user device, one of an electronic service request or registration request; transmitting to the user, over a network, a questionnaire; receiving responses to the questionnaire from the user device over the network; and executing an algorithm to assign the user to one or more clusters, wherein each of the clusters is defined by a data set of previously obtained responses corresponding to a particular character trait. The algorithm may compare the user's responses to the previously obtained responses; determine the frequency with which the user's responses overlap with the previously obtained responses; assign the user to the cluster having the greatest frequency of overlap; generate a list including at least one service provider based on the assigned cluster; transmit the generated list including at least one service provider to the user device over the network; and cause the generated list to be displayed to the user on the user device.

Yet another aspect of the disclosure provides a method including storing a list of service providers and clusters of community demographic information in one or more network-based non-transitory storage devices; receiving, over a network, user identifying information from a user device; storing the user identifying information in a standardized format in the one or more non-transitory storage devices; analyzing the user identifying information to match the user to at least one of the clusters; assessing one or more user risk factors based on the at least one of the cluster to which the user is matched; automatically generating a customized list of service providers based on the assessed one or more user risk factors, wherein the customized list is based on the user's assessed risk factors and the customized list is narrower than the stored list of service providers; and transmitting the customized list of service providers to the user device.

Yet another aspect of the disclosure provides a method for cross-channel communication. The method may include receiving a selected service category over a first communication channel associated with a platform; generating, by a machine learning model, available service providers based on the selected service category, a geographic location, and service provider availability information; rendering the available service providers; receiving a selected service provider of the available service providers; rendering a referral message associated with the selected service provider; embedding a correlation identifier in the referral message; transmitting the referral message over a second communication channel external to the platform; receiving a response from the selected service provider over the second communication channel; correlating the response with the referral message by extracting the correlation identifier; extracting text of the received response; and rendering the extracted text over the first communication channel.

Yet another aspect of the disclosure provides a computer-implemented system for generating a vulnerability tier. The system may include at least one processor; and a storage medium storing instructions that, when executed, configure the at least one processor to perform operations. The operations may include transmitting to a user device for display on a user interface, over a communication network, a questionnaire comprising questions that represent a plurality of categories; receiving responses to the questionnaire from the user device over the communication network; retrieving supplemental user information from one or more data sources; converting the responses and the supplemental user information; assigning, using a machine learning model, weights to the responses based on correlations among responses; computing base scores for the plurality of categories from the weighted responses using a hybrid scoring system; quantifying, using a combination function, compounding vulnerabilities across categories; applying the compounding vulnerabilities to the base scores to produce a composite vulnerability index; comparing the vulnerability index score to thresholds associated with vulnerability tiers to determine a vulnerability tier of a user; generating and transmitting a vulnerability tier record to the user on a graphical user interface, including an explanation of the determined vulnerability tier.

It is to be understood that the foregoing general description and the following detailed description are exemplary and explanatory only, and are not restrictive of the disclosed embodiments.

### BRIEF DESCRIPTION OF THE DRAWINGS

The accompanying drawings, which are incorporated in and constitute a part of this disclosure, illustrate various disclosed embodiments and, together with the description, serve to explain the disclosed principles. In the drawings:

FIG. 1 is schematic illustrating an exemplary system in accordance with embodiments of this disclosure.

FIG. 2 is an illustration of an exemplary questionnaire in accordance with embodiments of this disclosure.

FIG. 3 is a diagram illustrating an exemplary application of a machine learning algorithm in accordance with embodiments of this disclosure.

FIG. 4A is an illustration of an exemplary dataset in accordance with embodiments of this disclosure.

FIG. 4B is a graph illustrating an exemplary output of a clustering algorithm in accordance with embodiments of this disclosure.

FIG. 4C is a graph illustrating an exemplary output of a clustering algorithm in accordance with embodiments of this disclosure.

FIG. 5 is a flowchart illustrating an exemplary process in accordance with embodiments of this disclosure.

FIG. 6 is flowchart illustrating an exemplary process in accordance with embodiments of this disclosure.

FIG. 7 is an illustration of an exemplary questionnaire in accordance with embodiments of this disclosure.

FIG. 8 is a flowchart illustrating an exemplary process in accordance with embodiments of this disclosure.

FIG. 9 is a flowchart illustrating an exemplary process in accordance with embodiments of this disclosure.

FIG. 10 is an illustration of an exemplary user interface in accordance with embodiments of this disclosure.

FIG. 11 is an illustration of an exemplary user interface in accordance with embodiments of this disclosure.

FIG. 12 is an illustration of an exemplary user interface in accordance with embodiments of this disclosure.

FIG. 13 is an illustration of an exemplary user interface in accordance with embodiments of this disclosure.

FIG. 14 is an illustration of an exemplary user interface in accordance with embodiments of this disclosure.

FIG. 15 is an illustration of an exemplary user interface in accordance with embodiments of this disclosure.

FIG. 16 is a flowchart illustrating an exemplary process in accordance with embodiments of this disclosure.

FIG. 17 is an illustration of an exemplary table in accordance with embodiments of this disclosure.

FIG. 18 is an illustration of an exemplary table in accordance with embodiments of this disclosure.

FIG. 19 is an illustration of an exemplary table in accordance with embodiments of this disclosure.

### DETAILED DESCRIPTION

The systems and methods described herein address drawbacks of traditional social service platforms. For example, the described systems and methods simplify and optimize the user need assessment process by using machine learning-based weights and relationship associations to generate service provider recommendations. Such systems and methods may allow the service platform to more accurately match users to relevant service providers and improve the likelihood of users receiving adequate service while also improving the system's storage and processing power. Furthermore, service providers may have better opportunities to accurately service the users based on their particular needs.

Disclosed embodiments may provide dynamic referral generation and may develop and send referral messages with little user input needed, track requests and responses to requests at all stages of communication, and securely enable cross-platform communication, effectively streamlining, tracking, and securing communication between users and service providers. Further, the disclosed embodiments may generate a vulnerability tier and may retrieve service provider questions for a user, normalize the user's responses, and inputs the normalized responses into one or more machine learning models to map the user to a vulnerability tier (e.g., based on considering non-linear composite vulnerabilities), enabling flexibility in service provider questionnaires and logic, while maintaining comprehensive and adaptive mapping techniques.

Disclosed embodiments may improve the speed and functioning of computerized systems by reducing the number of service providers that must be evaluated during a search by defining a geographic area that limits the considered service providers and/or by defining criteria for service provides to be considered (e.g., one or more thresholds associated with a response times, acceptance rates, and/or frequencies of unanswered referrals.).

Disclosed embodiments may associate a correlation identifier with each referral, allowing the system to match service provider responses received across different communication channels with outgoing referral messages without having to scan message bodies. Such systems, methods, and computer-readable mediums improve accuracy in reconstructing message threads and reduce the processing required to manage communications across multiple channels.

Disclosed embodiments may utilize layered encryptions for communications. For example, the disclosed embodiments may encrypt communications with a user, back-end server (e.g., a server hosting a platform), and external systems (e.g., systems associated with the service providers). The disclosed embodiments may also encrypt sensitive data stored in a database. This approach provides security while providing communication with multiple third-party systems to obtain comprehensive data.

Disclosed embodiments may further improve the functioning of a platform and computer by storing multiple intake questionnaire question sets and evaluating information relevant to a user which question set is the most appropriate to present to the user. The disclosed embodiments may provide a schema-agnostic questionnaire, assign weights to questionnaire responses based on significance and inter-response correlations, and evaluate compounding vulnerabilities across service categories. Such systems, methods, and computer-readable mediums improve the ability of system to interact with multiple external service provider systems, and provide for identification and evaluation of nuanced aspects of a user situation, improving accuracy in assessing user need.

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions, or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

Some embodiments of this disclosure may involve systems and methods for assessing a customer's medical or behavioral health needs. Although embodiments of the current disclosure may be used to determine other needs. As used herein, a “customer” is any individual, group of individuals, business, or entity. For example, the customer may be a minor, adult, or an actor, e.g. a caregiver, parent, guardian, spouse, boss, social worker, physician etc., acting on behalf or in a representative capacity of another minor or adult. The customer may also be an entity, e.g., a hospital, treatment center, school, youth organization, place of worship, etc., acting on behalf or in representative capacity of one or more members of the entity. The term “need” as used herein, means any tangible or intangible good or service, that if met would improve the customer's health, behavioral, financial, environmental, educational, or socio-economic disposition. For example, the systems and methods disclosed herein may determine that a customer, such as a child, has a need for food or school materials (a tangible good) and therapy (intangible service). The term “customer” may be used interchangeably with the term “user”.

The embodiments disclosed herein may include a computing device. As used herein, a “computing device” is any computer, distributed computing system, server, or network of servers capable of receiving, processing, and transmitting data over a network, such as the internet. The computing device may include one or more processors, memory having executable instructions, and storage, as are known in the art. The computing device may be configured to receive an electronic service request from the user. The service request may be an inquiry for a particular type of service provider e.g., a physician or therapist, or a request for a service provider recommendation based on a need. In the latter scenario, the user might, for example, notice concerning behavior or signs of trauma, e.g., that a student is “acting out” or exhibiting signs of emotional trauma, and request a service provider recommendation to address the underlying cause of the behavior or trauma. The server may also be configured to receive an electronic customer registration request, whereby the customer requests to sign-up for the services disclosed herein. The requests may be received from the customer over a network.

In accordance with some embodiments, the customer may submit the request from a remote location using a customer device, e.g., a desktop, laptop, mobile phone, kiosk, personal digital assistant, tablet, smart TV, or other network-enabled device. The term “customer device” as used herein refers to a device used by the user, and is not meant to be limiting regarding ownership of such a device. The term “customer device” may be used interchangeably with the term “user device”. Both the user device and computing device are connected to a network, such as the internet, cellular network, local area network (LAN), metropolitan area network (MAN) or wireless area network (WAN), thus allowing communications to be transmitted between the computing device and the user device. The user device may include a service platform comprising a downloaded, installed, or web application that directs the user to a user interface when the user opens the application. For example, the platform may include a mobile application and/or a web-based application. The user may submit the request using the interface. In some embodiments, the user may launch a web browser and access the user interface by inputting a web address corresponding to the user service platform in the web browser. In some embodiments the user may receive a link via e.g., email or text message, that directs the user to the interface when clicked. The interface may be a graphical user interface including a plurality of selectable options.

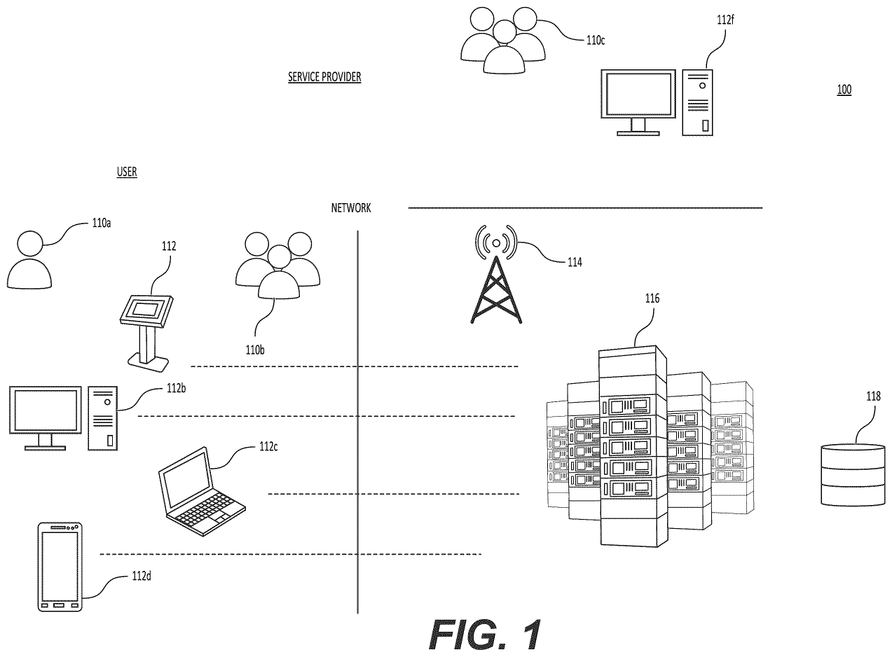

Upon receiving a request from the user, the computing device may transmit an electronic questionnaire to the user over the network. FIG. 1 illustrates an exemplary system in accordance with this disclosure. A user, e.g. individual 110a or entity 110b may use a network enabled user device 112a-d to submit a registration request to the service platform. The registration request may be submitted over a network 114, such as a cellular network, to one or more servers operating as computing device 116. Upon receiving the request, computing device 116 may send a questionnaire over network 114 to the user 110a/110b for completion. The user 110a/110b may complete all or a portion of the questionnaire and submit the responses to computing device 116 over the network 114 using the network-enabled device 112a-d. Computing device 116 may process the responses and store them in one or more databases 118 as discussed below. Computing device 116 may include one or more processors and at least one memory (e.g., a flash memory) to perform the disclosed processes. In some embodiments, computing device 116 may be associated with a third party that hosts a platform for performing one or more processes described herein. For example, computing device 116 may allow a third party to manage a platform, such as a more of a mobile application, web-based application, or cloud-based platform.

Computing device 116 may further communicate over a network to a plurality of service providers 110c through service provider devices 112f. Computing device 116 may comprise a plurality of different modules (e.g., program modules) and/or processors for executing different functions, such as generating questions, encrypting information, matching user(s) and provider(s), generating referrals, managing policies, a storing and analyzing data, and generating explanations.

As further detailed below, computing device 116 may select questions that the questionnaire consists of, encrypt information over the network and stored in the one or more databases 118, and match a user and a service provider using one or more machine learning models. Computing device 116 may generate a referral message to transmit to a service provider, may establish the rules that system 100 operates under and govern the components of system 100, and may store and analyze the data collected by system 100. Computing device 116 may develop explanations for outcomes within system 100 and may present the explanations to a user of the system (e.g., via user devices 12a-d).

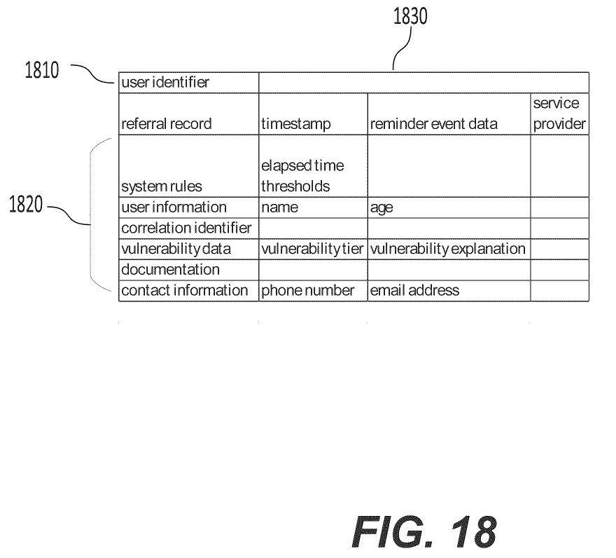

In some embodiments, computing device 116 may store and associate data in a plurality of searchable tables (e.g., as further illustrated in FIGS. 18-20 below) and/or in format(s) that allow for linking and searching data. These searchable tables may be in a local memory of computing device 116 or in the one or more databases 118. Searchable tables may include one or more of a service provider table, a user table, and a third-party table.

The service provider table may consist of data related to the service providers, which may include name, location, contact information (e.g., email address, phone number, etc.), and category of service. The service provider table may include text and objects to include in the referral message. The service provider table may consist of provider-specific attributes, which may include availability and supported languages. The service provider table may also consist of data related to contact means, which may be ways that the user can communicate with service providers. For example, contact means may include emails, SMS messages, and/or application programming interface (API) webhooks. The service provider table may store all available contact means of a service provider. In some embodiments, the service provider table may also store the preferred contact means of service providers. The service provider table may consist of service provider review and analytical information, which may be information used to evaluate the service providers. Service providers may be evaluated on their referral response times, time taken to provide services, referral acceptance rates, referral fulfillment rates, and/or service success rates. Review and analytical information may also include user feedback, historical user selections, and past ranks. In some embodiments, service provider review and analytical data may be collected and/or evaluated by computing device 116. In some embodiments, the service provider table may store service provider identifiers, which may be used by computing device 116 to identify user selections. Computing device 116 may periodically update the information stored in the service provider table.

The user table may consist of data related to a user, which may include name, other identifiers, location (e.g., GPS detected location), contact information, information associated with a user account, and information computing device 116 receives from external sources. The user table may also consist of referral messages and referral records, which may include information on past requests (e.g., service provider, timestamp). Other identifiers may include a correlation identifier and/or a verification credential. In some embodiments, other identifiers may be used to identify a user that does not log into an account. For example, computing device 116 may identify a user that does not log into an account with a temporary session identifier or a device-generated token. The temporary session identifier or device-generated token may be created when the user initially interacts with the service platform, signifying the beginning of a user session and may allow computing device 116 to associate actions like category selection, referral entry, and/or provider interaction with the user session. The user table may also consist of information computing device 116 uses to determine user eligibility for certain services, including information derived from the user device 112a-d and information provided by the user. Examples of eligibility information may include the user's age, household status, current employment situation, and living situation. The user table may also consist of documentation that identifies additional information about a user. Computing device 116 may use a user identifier to search the user table for information associated with a particular user.

The third-party records table may include authorized identifiers, such as user-specific-tokens, case numbers, and/or alternative reference values, and contact information for electronic health record (EHR) system and statewide automated child welfare information system (SACWIS). The third-party records table may include requests sent by computing device 116 to agencies. The third-party records table may include rules and policies, such as data-sharing rules and privacy policies, of agencies.

As further detailed below, the questionnaire may include a variety of questions intended to solicit information about the user's identification and needs. For example, some questions may inquire as to the user's name, address, date of birth, and other demographic information, while other questions may be tailored to assess a particular need. For example, health-related questions may inquire as to a user's health history (e.g. chronic conditions, height, weight, allergies, surgeries or other procedures, medications, or other health indicative information), whether and how often the user sees a physician (PCP, ophthalmologist, dentist, or other medical specialist). Socio-economic related questions may inquire as to the user's employment status, income, family size, educational background, whether the user ever received social welfare services, whether the user was ever arrested, convicted of a crime, or imprisoned, or other questions indicative of the user's socio-economic status. Behavioral-related questions may inquire as to whether the user was ever arrested, convicted of a crime, imprisoned, abused, abandoned, used or exposed to drugs and alcohol, whether the user would describe his/her upbring as nurturing or violent, or other questions that may be determinative of emotional or physical trauma.

If the user is a business or other entity, the questionnaire may ask the user if it would like to provide services to other users associated with the platform. For example, the questionnaire may ask a pediatrician completing the questionnaire on behalf of a patient whether the pediatrician would like his services and/or practice to be added to the platform's network of healthcare providers. If the provider agrees to be listed, computing device 116 may store the provider's demographic information in a database. In some embodiments, the provider's demographic information may be stored in the service provider table. In this way, the platform may compile a database of confirmed platform affiliates, that may be recommended to other users, as will be discussed in further detail below.

In some embodiments the system is adaptive such that the questionnaire automatically generates questions based on responses to previous questions. For example, if a user responds that he or the person on whose behalf he is completing the questionnaire is 10 years of age, the questionnaire may not present questions related to employment history and income, but may present questions related to school attendance, school grades, and school disciplinary history. The questions may solicit a binary response, e.g. “yes” or “no”. In other embodiments the questions may be multiple choice, and offer a variety of responses, e.g., “yes”, “sometimes yes”, “never”, “likely”, “unlikely”, “somewhat likely” or “somewhat unlikely.” In some embodiments, the questions may solicit a response in the form of a numerical range, e.g., 1-10, or be open-ended, so that the user can provide a narrative response. The questionnaire may include any combination of question types and at least two questions.

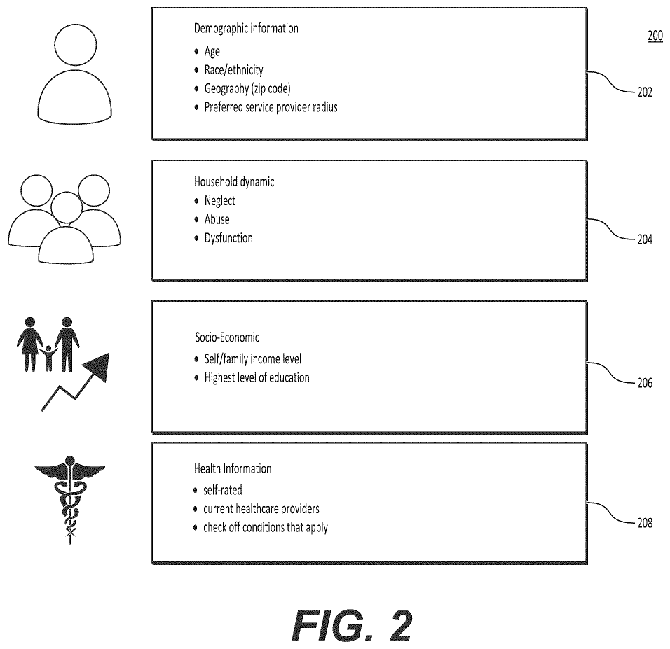

FIG. 2 illustrates an exemplary questionnaire in accordance with embodiments of this disclosure. Questionnaire 200 may include subsections 202-208, each of which focuses on a particular category. For example, subsection 202 may prompt the user for demographic information such as age, race/ethnicity, zip code, and a preferred service provider radius. The preferred service provider radius may be used to narrow the list of service providers so that they are situated within the prescribed radius. In some embodiments, the system may use geolocation to ascertain the user's location and automatically narrow the list of service providers based on a preferred radius. For example, the system may determine the user's location using GPS, network triangulation, Wi-Fi positioning, Bluetooth, the user device's IP address (and other device identifiers), or other methods known or considered obvious to one of ordinary skill in the art.

Subsection 204 may prompt the user for information relating to the user's household dynamic. For example, subsection 204 may ask whether the user has ever been physically or emotionally neglected, abused (e.g., bodily, psychological and or sexual abuse), and/or the nature of any household dysfunction (e.g., whether members of the household, including the user suffer from substance/alcohol abuse or violence). If the user is a minor, the questionnaire may further inquire as to whether the minor's parents are separated. Subsection 206 may prompt the user for socio-economic information. For example, subsection 206 may inquire as to the user's income level and education. The income level may be reported in quantitative terms, e.g., household salary or in subjective terms, e.g., “below average”, “average”, or “above average.” If the user is a minor, subsection 206 may inquire as to the highest education level of the user's parents. Subsection 208 may inquire as to the user's health information. For example, subsection 208 may prompt the user to rate their overall health (e.g., “below average”, “average”, or “above average”), to provide a list of current healthcare providers (e.g., PCP, dentist, ophthalmologist, cardiologist, etc.), and to check off any health conditions (e.g., migraines, diabetes, hypertension, glaucoma, heart disease, cancer) that might apply. The user may also be prompted to provide basic health statistics, e.g., weight and height.

Although multiple subsections are illustrated, one of skill in the art will appreciate that a single subsection or grouping of subsections may be presented to the user. In accordance with some embodiments, the questionnaire may be customized to meet the needs of a particular entity. For example, an entity such a behavioral health specialist may upload a preferred questionnaire into the platform. The platform may standardize the form to ensure that the response format is compatible with the machine-learning algorithms discussed below. For example, the platform may configure the questions so that all responses are submitted in a “true/false” format. In some embodiments, the system automatically customizes the questions based on the system's analysis of the user's responses to previously answered questions. The questions may get smarter with each question answered thereby streamlining the questionnaire process. In some embodiments, the system customizes or adapts subsequent questions provided in the questionnaire in real-time, based on the system's analysis of the user's responses to previously answered questions. For example, based on the user's answers to the first three questions, the system may generate the next three questions in real-time.

Upon responding to the questionnaire, the user may submit the responses to computing device 116 for processing. In some embodiments, the user may submit the response by selecting “send” or “submit” on the platform website or application. Although it is preferable for the user to complete the questionnaire prior to submitting, completion is not necessary for submission or processing.

Computing device 116 may be configured to process the responses when received. In accordance with some embodiments, the processor may execute a machine learning algorithm on the responses to extract associations and determine a relationship between at least two of the questions based on the user's response. The algorithm may be a supervised learning algorithm such as Naïve Bayes, K-Nearest Neighbors (KNN), or other classification algorithm configured to predict a categorical outcome based on a given sample. The algorithm may also include a Calculation and Regression Tree (CART), Linear Regression, Logistic regression, or other regression algorithm configured to predict a quantitative outcome of a given sample.

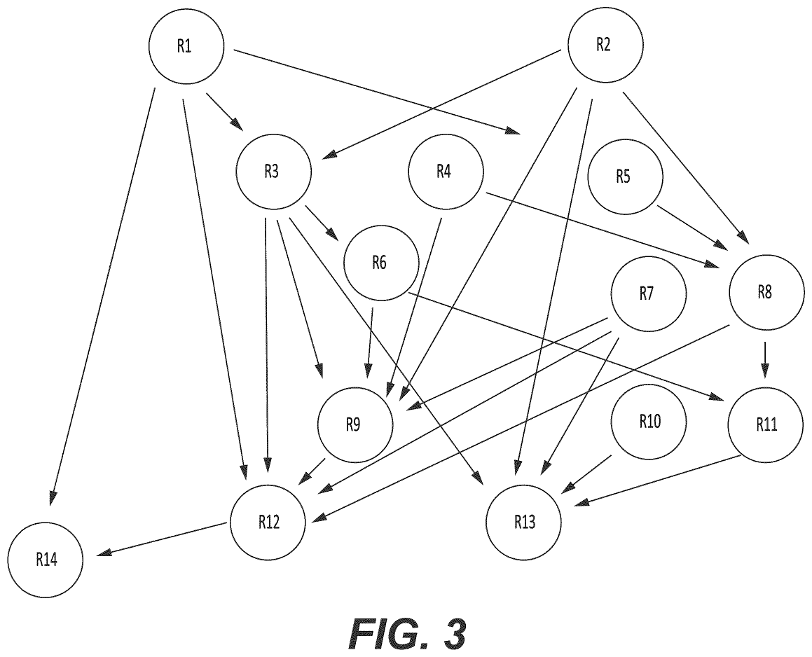

For example, a Naïve Bayes algorithm may be executed on a set of user questions and responses to determine how a response to one question relates to a response to another question. Relationships may be assessed for all questions Q₁ through Qₙ and responses R₁ through Rₙ in an iterative style to determine the relationship between any one question/response and any or all other questions and responses to generate a relationship map whereby a user's response to one question is predictive of how that user will respond to another question. FIG. 3 is an exemplary graphical representation of a relationship map.

Regarding FIG. 3, R1-R14 represent user responses to questions Q1-Q14. R₂ is an affirmative response that is predictive of affirmative responses R₃, R₈, R₉ and R₁₃. Because R₂ is highly predictive, the question it corresponds to is useful for determining how a user might answer other questions. This predictive relationship may be assigned a quantitative value between 0 and 1. The closer the value is to 1, the more predictive the question. In accordance with embodiments of this disclosure, the algorithm may designate questions having a relationship value greater than 0.5 as critical questions because of their predictive potential. Other values, e.g., 0.6, 0.7, 0.8 and 0.9 (or values therebetween) may be used as the critical value threshold. The algorithm may then determine one or more user needs based on the user's responses to critical questions.

In some embodiments, the processor may be configured to execute multiple machine learning algorithms. For example, a Naïve Bayes algorithm may be executed on the responses to predict a user need and a Linear Regression algorithm may be executed to determine a probability that the user will benefit if the need is addressed by a service provider. In some embodiments an unsupervised algorithm may be executed on the responses in addition to one or more supervised learning algorithms to determine relationships between the users. The unsupervised algorithms may include, for example, Apriori, K-means, PCA or other associations, clustering, or dimensionality reduction algorithms. For example, a clustering algorithm, such as K-means may be executed on a data set of user responses to group users having similar response patterns into clusters. The Naïve-Bayes algorithm may then be executed to predict the need of a user based on the cluster to which the user is assigned. Exemplary embodiments illustrating an application of the algorithm will be discussed in further detail below.

Upon determining one or more user needs, the computing device may match the user to a service provider suitable for addressing the need. The service provider may be selected from a publicly available directory of confirmed platform affiliates, as discussed above. In some embodiments, restrictions may be placed upon the matched service provider. For example, a geographic restriction may be used so that the recommended provider is located within, for example, 5 miles of the user's home address or determined device location. The processor may be further configured to generate a list of the critical questions and store them in one or more databases 118. The critical questions may be categorized by question type. For example, critical questions may be critical behavioral questions, critical health questions, critical financial questions, etc. In some embodiments, the generated list may be transmitted to the recommended service provider so that the service provider knows which questions to discuss with the user.

Embodiments disclosed herein may use a computer-implemented method to generate a list including at least one service provider. The service provider may be a confirmed platform affiliate or selected from a public directory as discussed above.

An electronic service request or registration request may be submitted over a network to the service platform from a user on a user device. The device may include a desktop, laptop, mobile phone, kiosk personal digital assistant, tablet, smart TV, or other network-enabled device, as discussed above. A questionnaire may be transmitted, in response to the request, to the user device over the network. The questionnaire may be transmitted from a computing device as discussed above. In accordance with some embodiments, the questionnaire may be downloaded on the user device and uploaded to the platform upon completion. In some embodiments, the questionnaire may be completed through an application operating on the user device. In some embodiments, the questionnaire may be completed through the platform's website, which is accessible through a web browser on the user device. The user may respond to and submit the questionnaire over the network to the computing device.

The computing device may be configured to store each of the responses in a database with other previously obtained user responses for further processing. In this way, the computing device may analyze each set of user responses by, e.g., comparing the response of one user against all sets of previously obtained user responses. In accordance with some embodiments, the computing device may execute a machine-learning algorithm to analyze the responses to determine a relationship between one set of user responses and one or more other sets of user responses in the database. The machine learning algorithm may compare the user's responses to the previously obtained responses and determine the frequency with which the user's responses overlap with the previously obtained responses. In accordance with some embodiments of the disclosure, the algorithm is a clustering algorithm, such as a K-means, hierarchal clustering, mean-shift clustering, Density-Based Spatial Clustering of Applications with Noise (DBSCAN), Expectation-Maximization (EM) Clustering, or other clustering algorithm as would occur to one having ordinary skill in the art.

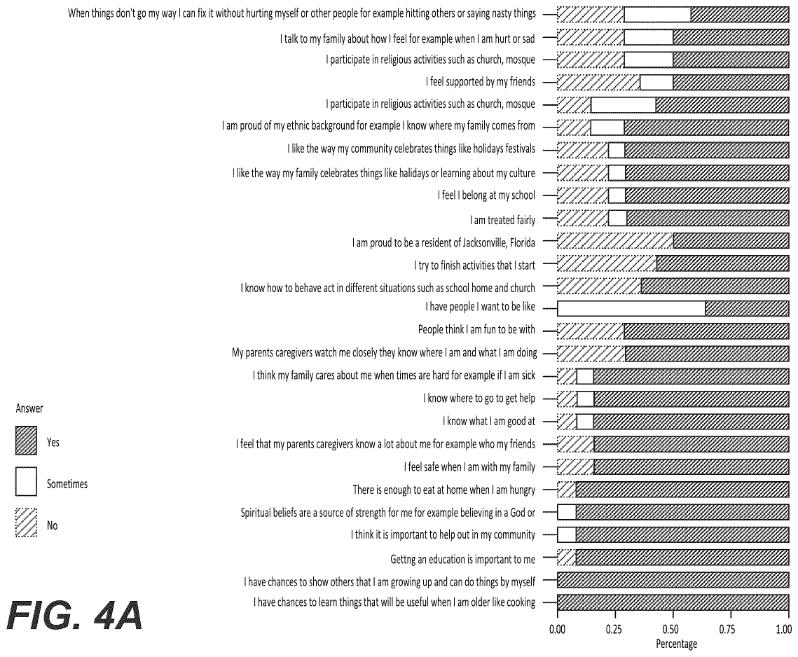

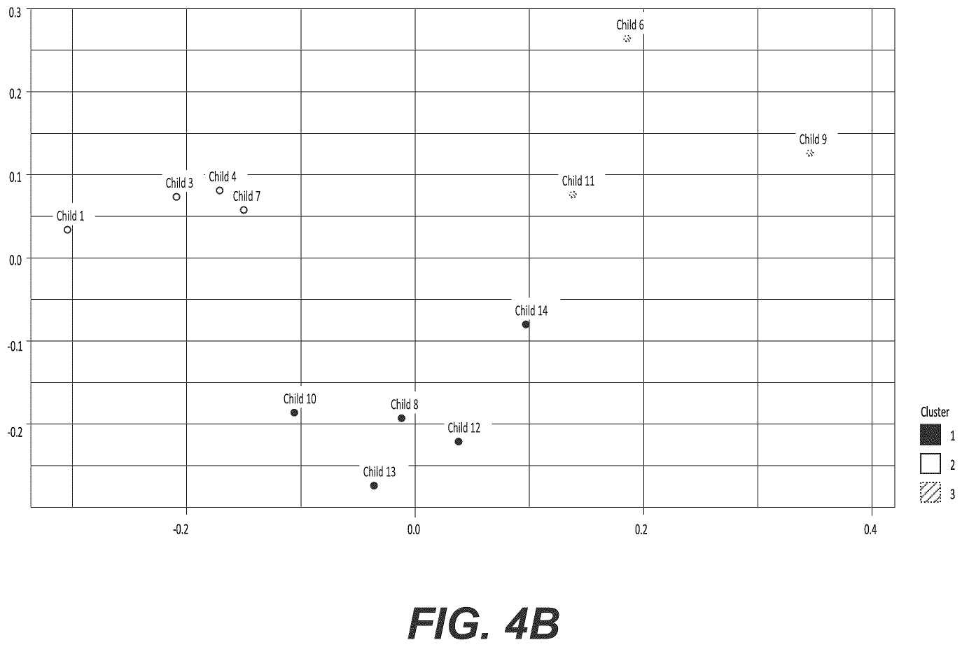

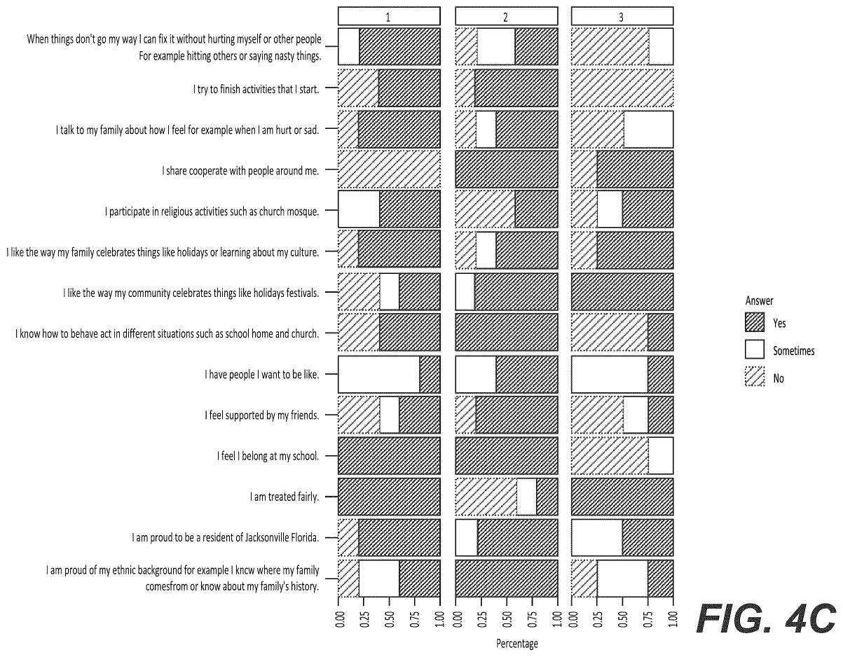

FIG. 4A is an exemplary chart illustrating the frequency of user responses within a database of user responses. For each question presented, the user had the option to respond “yes” depicted in black, “sometimes” depicted in light grey or “no” depicted in dark grey. The frequency of each response is measured across all users (i.e., how often all users respond “yes” to a question) and conveyed as a percentage. The machine-learning algorithm may determine, based on the response and comparison, that the user shares certain traits with other users, and assign the user to a cluster corresponding to the greatest frequency of overlap. FIG. 4B illustrates an output of the clustering algorithm in which each user is assigned to one of three clusters based on the responses received. For example, Child 6, Child 9, and Child 11 are assigned to Cluster 3. FIG. 4C illustrates another output of the clustering algorithm, wherein for each cluster 1-3, the question posed, and frequency of “yes”, “sometime” and “no” responses is provided.

The cluster may have an identifier describing a particular trait. The trait may be related to health, behavior, or another category. For example, if the questionnaire relates to a behavior assessment, the identifiers may describe behavioral traits. Users whose responses overlap may then be assigned to clusters associated with the particular behavioral trait.

A behavioral application will now be discussed with respect to FIG. 4B-C. Questions may inquire as to how a child user responds to certain behavioral situations. For example, one question may read “I try to finish things when I start them” and prompt the user to select “yes” “sometimes” or “no.” Other questions may read “I share with people around me”, “I have people I want to be like”, “I feel I belong at my school”, “I am treated fairly”, “I talk to my family about how I feel”, etc. Upon receiving responses to the question, the machine-learning algorithm analyzes the responses and assigns the child users to clusters based on how the child users respond to the questions. As seen in FIG. 4B, fourteen user datasets are processed, and three clusters (Cluster 1, Cluster 2, and Cluster 3) are generated. The breakdown of responses for each cluster is illustrated in FIG. 4C. In this example, the child users in cluster 1, responded (predominantly) “yes,” but also sometimes “no” to the question “I talk to my family about how I feel” while the child users in cluster 3 never responded “yes” and the child users in cluster 2 respond (predominantly) “yes” but also “sometimes” or “no” in equal amounts. The clusters may be labeled with an identifier describing the behavioral traits of the child users in that cluster. For example, the child users in cluster 1 tend not to share or cooperate with others and do not have many people they want to be like. This cluster may be labeled “unsocial.” The child users in cluster 2 like to share but generally do not feel that they are treated fairly and, more often than not, do not participate in religious activities; thus, cluster 2 may be labeled “Unfair.” The child users in cluster 3 tend to not talk to their families about their feelings and tend to harm themselves or others; thus, this cluster may be labeled “Hurt.”

Using the assigned cluster, the computing device may generate a list including at least one service provider who specializes in treating users having the behavior trait associated with the cluster. The list may be transmitted to the user device. The questions and responses corresponding to the assigned cluster, i.e., those questions and responses that caused the user to be assigned to the cluster, may be transmitted to the service provider and/or user. In this way the service provider may know which questions and responses may be particularly useful to consider or discuss upon seeing/treating the user.

In some embodiments of this disclosure, the platform may store and update responses to create a more intuitive platform. A list of services providers and user clusters may be stored in one or more network-based non-transitory storage devices, such as a server or other computing device. Users provided identifying information, such as the responses discussed and described above, may be stored in a repository in the one or more non-transitory storage devices in a standardized format. The information may be submitted remotely over a network through a graphical user interface of a user device. The user-provided responses may be analyzed using one or more of the machine learning algorithms discussed above. For example, the information may be processed using a clustering algorithm to match the user to at least one of the clusters or the information may be processed using a Naïve Bayes-algorithm to determine a user need, as discussed above. Alternatively, both algorithms may be executed simultaneously or successively on the information. For example, a clustering algorithm may be executed to assign the user to a cluster and a naïve bayes algorithm may be executed after the user has been assigned, to determine a user need or one or more user risk-factors based on the responses corresponding to the assigned cluster. The database may be automatically updated to include each successive set of user responses so that the algorithm continuously updates the clusters and/or needs based on the universe of user responses provided. In this way, the platform is able to learn about its users and provide increasingly useful service provide recommendations.

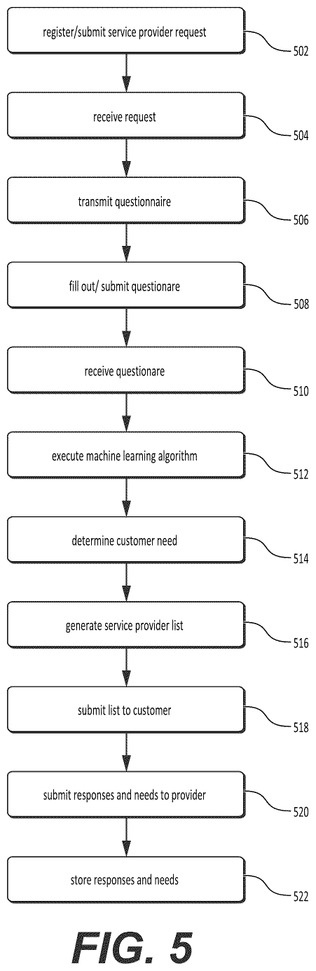

FIG. 5 describes an exemplary method of determining a user need in accordance with embodiments of this disclosure.

At step 502, a user 110a/110b submits a registration or service provider request using a user device 112 over a network 114. The user may be an individual seeking assistance on their own behalf or an individual or entity seeking assistance on another's (e.g., child, parent, patient) behalf. At step 504, computing device 116 receives the request and transmits a questionnaire to the user device. The questionnaire may be embodied in a standard format such as questionnaire 200 illustrated in FIG. 2. Alternatively, the questionnaire may only contain questions relating to a specific category 202-208, or a combination of two or more categories. The questionnaire may also be acquired from an entity and uploaded into the platform for presentation to the user. For example, the questionnaire may be similar to the intake form a particular treatment facility or social services group, or be modeled after another assessment form, e.g., Adverse Childhood Experience (ACE) questionnaire. At step 506, computing device 116 transmits the questionnaire over the network to the user device. In accordance with some embodiments, steps 502-506 may be optional. For example, the questionnaire may be available on, e.g., an interface of the device 112 and completed without submitting a request. The questionnaire may be filled out and uploaded into the platform and submitted at step 508. Computing device 116 may receive the responses to the questionnaire at step 510 and execute a machine-learning algorithm at step 510.

The algorithm may be a clustering algorithm and/or predictive algorithm as discussed above. For example, a K-means clustering algorithm may be executed on the responses to assign the user to at least one cluster. The cluster may be used to assess the user's needs and match the user with a service provider at step 514. Alternatively, a Naïve Bayes-algorithm may be executed on the responses to generate a list of critical questions and determine the user's need(s). Alternatively, both algorithms may be executed simultaneously or successively on the information. For example, the clustering algorithm may be executed on the responses to assign the user to a cluster and a naïve bayes algorithm may be executed after the user has been assigned, to determine a user need or one or more user risk-factors based on the responses corresponding to the assigned cluster. A regression algorithm may also be executed determine a likelihood that the user will benefit from having his/her needs treated/met by a service provider.

At step 516, computing device 116 may generate a list of one or more service providers for treating/meeting the user's need(s). The list may be transmitted to the user device at step 518 and the list of critical questions, user responses and need(s) may be transmitted over the network to the user device and/or service provider at step 520. All responses and needs may be stored in a database of computing device 116. Steps 512-522 may be repeated for each set of responses received. Once the user is successfully registered with the platform, the user may receive an account login or other credentials. The user may visit his/her platform account to track matches, update responses to the questionnaire, or provide feedback for services received.

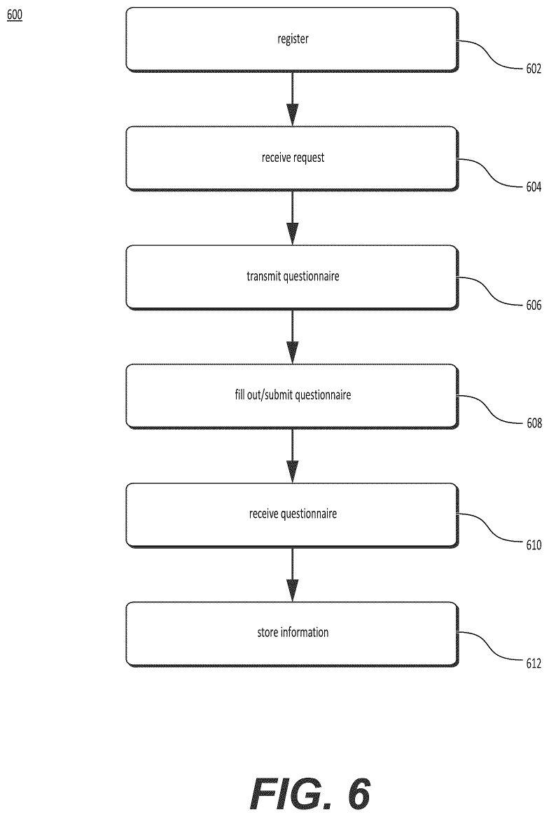

FIG. 6 illustrates a method of building a service provider network. Steps 602-610 are substantially similar to steps 502-510 above except for the contents of the questionnaire.

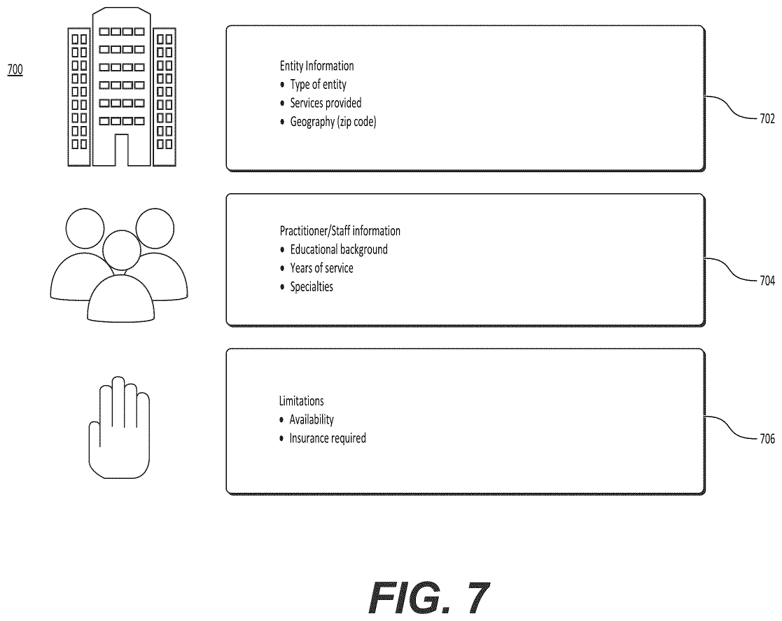

Here, the questionnaire may be crafted to identify the types of services the provider can provide and whether there are any limitations to that service. The questionnaire may be modeled after exemplary questionnaire 700, illustrated in FIG. 7. Questionnaire 700 may include subsections 702-706. Subsection 702 may prompt the user for entity information such as entity type, services provided, or zip code. The entity type may be selected from a list of options, e.g., social worker, allergist, physical therapist, shelter, doctor, etc., or may be typed into a field. The services provided may be selected from a list of options corresponding to the entity type or provider, or typed into a field. Alternatively, the provider may link a webpage to the provider's website and the platform may determine the services provided based on the link. Subsection 704 may prompt the provider for information relating to the provider's staff or practitioners. For example, the provider may list the staff member's educational background (e.g., Doctor of Dental Surgery-DDS), time of service (e.g., 8 year) and specialties (e.g. braces and orthodontics). Alternatively, the provider may upload a link to the staff member's webpage biography and the platform may determine the aforementioned credentials based on the text of the biography. Subsection 706 may prompt the user for entity limitations. For example, subsection 706 may inquire as to the entity's next available appointment and whether insurance is required. If insurance is required, subsection 706 may further inquire as to the type of insurance needed. In some embodiments, the system automatically customizes the questions based on the system's analysis of the provider's responses to previously answered questions. The questions may get smarter with each question answered thereby streamlining the questionnaire process. For example, if the provider indicates they are a dentist, the selectable option under “services provided” may be limited to relevant dental services, e.g., prosthodontics, oral surgery, braces, etc.

The questionnaire responses may be submitted to computing device 116 at 610 and stored at step 612. Once the provider is successfully registered with the platform, the provider may visit his/her platform account to track matches and service requests, update questionnaire responses, and/or request to be removed from the provider network.

Alternatively, a provider may be added to the network while the provider is requesting services on another's behalf. For example, if the user of FIG. 5 is a business or other entity, the questionnaire may ask the user if it would like to provide services to other users associated with the platform. For example, the questionnaire may ask a pediatrician completing the questionnaire on behalf of a patient whether the pediatrician would like his services and/or practice to be added to the platform's network of healthcare providers. If the provider agrees to be listed, the computing device may store the provider's demographic information in a database. In this way, the platform may compile a database of confirmed platform affiliates, that may be recommended to other users.

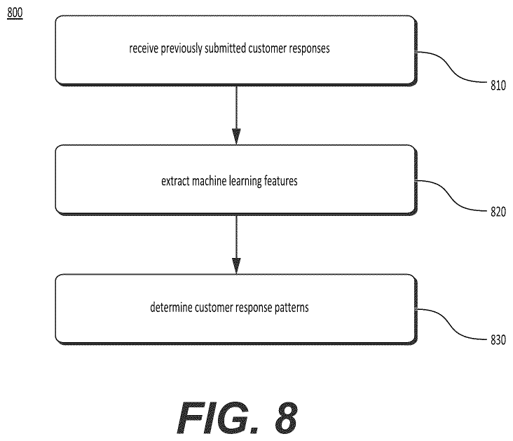

Referring now to FIG. 8 in some embodiments, the system may train the machine-learning algorithm using user questions and responses. In this way, each iteration of questions and responses may cause the clusters and/or critical questions to be automatically updated so that the platform becomes smarter with each user submission. For example, at step 810 of process 800, system 100 may train a machine learning model using information on previously submitted user responses. In some embodiments, the information on previously submitted responses includes information on user behavioral, health, or socio-economic responses. At step 820, system 100 may be configured to extract machine learning features from the user responses. In some embodiments, these features can be extracted by inputting previously obtained user responses into a first convolutional neural network. The first convolutional neural network can be configured to output the machine learning features. System 100 can extract user response patterns from the machine learning features. In some embodiments, these features can be extracted by inputting the machine learning features into a classifier, such as a second convolutional neural network. In some embodiments, the second convolutional neural network can be distinct from the first convolutional neural network. The second convolutional neural network can be configured to output indications of the response patterns. At step 830, system 100 can be configured to determine a user need or a data point for a user profile based on the extracted machine learning features. For example, system 100 may be configured to determine the user's behavioral, health, or socio-economic needs based on the machine-learning model, and store the determined needs as part of a profile on the user's account.

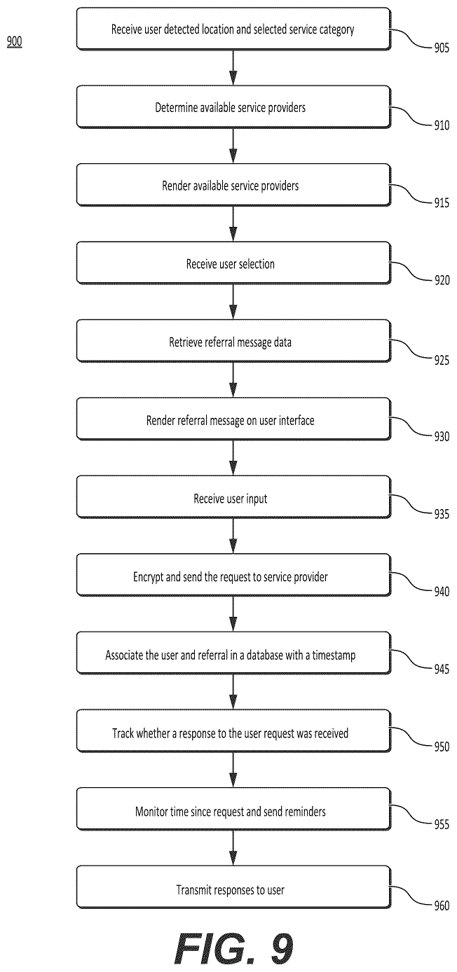

FIG. 9 describes an exemplary method 900 for generating and tracking a referral message in accordance with embodiments of this disclosure. At step 905, computing device 116 receives a user location, a selected service category, and/or user eligibility information. In some embodiments, computing device 116 may receive a user location based on location tracking services of the user device 112a-d. For example, computing device 116 may request permission from the user to access location tracking services of the user device 112a-d. Upon receiving permission from the user, the user device 112a-d retrieves geographic location data from a location service of the operating system (e.g., GPS, Wi-Fi positioning, or cellular triangulation) and transmits the location data to computing device 116 over the network. While in other embodiments a user may enter their location information on user device 112a-d. For example, computing device 116 may present a prompt to the user requesting the user to enter or select their location on the user interface. Upon receiving a user input, computing device 116 may receive the location information. Computing device 116 may store the user location in the user table.

In some embodiments, computing device 116 may receive a selected service category. Service categories may include but are not limited to food, legal, housing, employment, healthcare, clothing, financial, youth, transportation, and education. For example, computing device 116 may prompt the user to select a service category via user device 112a-d. The prompt may include presenting a set of selectable service category elements. In some embodiments, the prompt may present all available service categories for user selection. The user may select (e.g. tap or click) a desired graphic element which may trigger transmission of the selected service category to computing device 116.

In some embodiments, computing device 116 receives additional information from user device 112a-d. Additional information may include and/or indicate user eligibility for certain services. Computing device 116 may determine user eligibility based on a user's age, geographic location, household status, program-specific requirements, and/or prior participation in related services. For example, computing device 116 may determine the user is eligible for a service because the user indicated that their household meets the requirement of the service to be a household with dependent children. In an alternative example, computing device 116 may determine the user is ineligible for a service because the user has reached a limit on a number of times a service can be received or because a service requires prior completion of another service.

In some embodiments, computing device 116 may determine user eligibility information for certain services based on information available from the user device 112a-d, such as information from the device's location services, time zone, regional settings, and language preferences. For example, computing device 116 may request permission from the user to access the language preference configured in the settings of the user device 112a-d. Computing device 116 may determine if a user is eligible for a service if the language preference matches one or more languages supported by the service provider. Alternatively, computing device 116 may determine user eligibility for certain services based on information provided directly by the user. Computing device 116 may receive the information from the user from user responses to prompts presented to the user. For example, computing device 116 may provide one or more prompts asking the user to input or confirm age range, current living status, and/or employment status. In some embodiments, the information requested in one or more prompts may correspond to attributes required by a service provider's policies. One or more prompts may be presented on a user interface of the user device 112a-d.

In some embodiments, computing device 116 may pull eligibility information based on the user's account details and/or link received eligibility information to a user. For example, computing device 116 may retrieve the age of a user from the user's account details, which are stored in the user table, to determine if the user is eligible to participate in a service that requires the participant to be at least 18 years of age. The age of the user may have been previously obtained by computing device 116 and stored in the user table when the user created an account on the service platform. In an alternative example, computing device 116 may store received eligibility information in the user table.

In some embodiments, computing device 116 may determine user eligibility for certain services without requiring the user to create an account. For example, computing device 116 may identify a user that does not log into an account with a temporary session identifier or a device-generated token. The temporary session identifier or device-generated token may be created when the user initially interacts with the service platform, signifying the beginning of a user session and may allow computing device 116 to associate information obtained from sending one or more prompts to the user or information available from the user device 112a-d with the user session. In some embodiments, the user may create an account afterwards, at which point computing device 116 may link previously gathered eligibility information associated with the user session to the account of the user.

The selected service category, user location, and/or user eligibility information may be received over a first communication channel associated with the service platform, which may be a channel that sends information between computing device 116 and a user device 112a-d. In some embodiments, all of the selected service category, user location, and user eligibility information are received over the first communication channel. In other embodiments, a portion of the selected service category, user location, and user eligibility information is received over the first communication channel.

At step 910, computing device 116 determines available service providers. Computing device 116 may determine the available service providers based on the selected service category, user location, user eligibility information, other user information collected in step 905, and/or service provider information. In some embodiments, computing device 116 may determine available service providers based on comparing user information collected and/or retrieved in step 905 with pre-stored service provider information (e.g., stored in a service provider table). In some embodiments, computing device 116 may compare service provider information with other pre-stored criteria. For example, computing device 116 may determine available service providers based on their availability windows overlapping with a user availability or pre-stored requirement and/or based on the service provider responsiveness meeting a predetermined threshold. Computing device 116 may identify available service providers by searching for providers that offer services in the selected service category within a defined geographic boundary. The defined boundary may include a threshold distance from the user location, a region that the user is located in, a location polygon of the user, or a geographic boundary of the user. For example, if health is the selected service category, computing device 116 may search for and identify health providers within a 10-mile radius of the user's location.

In some embodiments, computing device 116 may determine available service providers using a machine learning model to identify and rank service providers based on their correlation to the user. In some embodiments, computing device 116 may retrieve a subset of service providers for analysis by its machine learning model. For example, computing device 116 may determine available service providers based on service provider availability information, a geographic boundary, or other determinations described above. Computing device 116 may filter a service provider table for the subset of service providers and/or otherwise tag or identify the service providers for analysis by its machine learning model.

Computing device 116 may retrieve input data for the machine learning model from the user table and the service provider table. Input data may include user detected location, selected service category, and eligibility information. Input data may also include historical interaction data, outcome data, and/or provider specific attributes. Historical interaction data may refer to prior engagement activity between users and service providers on the platform, including referral attempts, user selections of service providers, communication channel usage, referral abandonment events, escalation history, repeat contact attempts, and/or other behavioral interaction records generated by computing device 116. Historical interaction data may reflect engagement behavior between users and providers. Outcome data may include information reflecting provider performance, including acceptance and/or fulfillment frequency, normalized or average response times, successful assistance rates from prior referrals, and/or other performance indicators. Provider-specific attributes may include information describing the characteristics, capabilities, and operating parameters of service providers. Provider-specific attributes may include availability and supported languages.

In some embodiments, computing device 116 may retrieve external data as input data for its machine learning model. External data may refer to generalized information retrieved by computing device 116 that is external to the service platform. Computing device 116 may retrieve external data through permitted interfaces or verified publicly accessible datasets. For example, external data may include service hours, holiday closures and third-party review metrics of service providers. In some embodiments, computing device 116 may normalize the external data before inputting it into the machine learning model. Normalizing the data may include converting and/or reformatting the data to a format suitable for the machine learning model and removing missing, false, or inconsistent data. For example, computing device 116 may reformat external data describing service hours or holiday closures into a common, pre-stored internal schedule representation.

Computing device 116, via the machine learning model, may evaluate the input data to identify available service providers and assign weights to the identified available services providers. The weights may represent a likelihood of meeting the user's needs. For instance, the machine learning model may assign higher weights to providers who historically respond promptly or who have demonstrated greater success in addressing the selected service category of need while the machine learning model may exclude or assign lower weights to providers who have been non-responsive for extended periods of time or infrequently fulfill requests. Computing device 116, via the machine learning model, may evaluate the input data to compute composite provider scores. A composite provider score may represent a calculated likelihood of a service provider meeting the user's needs. For instance, computing device 116 may assign higher composite provider scores to providers who historically respond promptly or who have demonstrated greater success in addressing the selected service category of need while computing device 116 may exclude or assign lower composite provider scores to providers who have been non-responsive for extended periods of time or infrequently fulfill requests. Computing device 116 may compute composite provider scores using a mathematical algorithm that combines the outcome data, historical interaction data, and provider-specific attributes. For example, computing device 116 may compile the outcome data, historical interaction data, and provider-specific attributes into an outcome value, interaction value, and attributes value, respectively. Consequently, outcome values may represent provider performance information, interaction values may represent prior engagement behavior between a user and service provider, and attributes may represent eligibility and compatibility factors associated with the provider. Computing device 116 may compute the composite provider score by calculating a sum of the outcome value, interaction value, and/or attributes value. Computing device 116 may also apply weighting factors to outcome values, interaction values, and attributes values to compute the composite provider score. For example, computing device may apply weighting factors to increase or decrease the effect of the outcome value, interaction value, and/or attributes value on a composite provider score. In some embodiments, the weighting factors may be predetermined. For example, the weighting factors may be predetermined based on user information and/or by an administrator of the platform. In some embodiments, the weighting factors may be configurable. For example, the weighting factors may be adjusted to reflect specific requirements of an administrator of the platform. In some embodiments, computing device 116 may dynamically adjust the weighting factors based on the machine learning model and/or performance metrics.

In some embodiments, the machine learning model may utilize a supervised algorithm based on historical input data and service provider outcomes. Computing device 116 may generate training data for the machine learning model from prior referral records stored in user tables and information stored in service provider tables. In some embodiments, computing device 116 may construct a training instance for at least one prior referral by extracting normalized user information (e.g., location, selected service category, eligibility information), historical interaction data, and provider-specific attributes (e.g., availability, supported languages, service area compatibility, etc.) and using the information to form an input vector associated with the specific referral event. Computing device 116 may derive an outcome label corresponding to the input vector from outcome data (e.g., acceptance and/or fulfillment frequency, normalized or average response times, successful assistance rates from prior referrals, and/or other performance indicators) associated with the referral record. In some embodiments, computing device 116 may compute the outcome label as a weighted combination of outcome metrics. For example, computing device 116 may sum values representing a fulfillment rate, a normalize response time, and a successful assistance rate to calculate the outcome label. Computing device may assign weighted coefficients to the values representing the various outcome metrics. Computing device 116 may pair the input vectors and outcome labels based on association with a prior referral record and aggregate the pairs to form a labeled training dataset for the machine learning model. The labeled training dataset may enable the model to learn relationships between combinations of user information, provider attributes, and interaction features and observed referral outcomes that may be applied when computing device 116 computes composite provider scores. Computing device 116 may periodically retrain the machine learning model as computing device 116 accumulates outcome data and other input data. Computing device 116 may anonymize information that identifies a user before using the information to train the machine learning model. In some embodiments, computing device 116 may train the machine learning model using anonymized data of a plurality of users across system 100.

Computing device 116 may generate a list of service providers ordered and/or ranked based on a predicted best fit for the user, reflected in the composite provider score assigned to the service providers. For example, computing device 116 may order and/or rank service providers assigned higher composite provider scores higher on a list, which may indicate a higher predicted suitability for addressing the user's identified needs, while computing device 116 may order and/or rank service providers assigned lower composite provider scores lower in the list.

In some embodiments, computing device 116 may store the weights and composite provider scores assigned to the service providers in the service provider table. In some embodiments, computing device 116 may store the list of service providers (and/or a subset of the list, such as a top 5 providers) and their associated ranking in the user table.

Computing device 116 may dynamically update the information used to determine available service providers, such as the inputs for is machine learning model. For example, in some embodiments, computing device 116 may use user selections, user feedback, and service provider response data to determine and rank available service providers. Computing device 116 may track and analyze user selections and user feedback, which may include ratings, and/or comments provided by a user. For example, a service provider that receives favorable feedback from a user may receive a higher ranking in a subsequent instance of computing device 116 determining available service providers while a service provider that the user identifies as unsatisfactory may receive a lower ranking or be excluded in a subsequent instance of computing device 116 determining available service providers. Computing device 116 may store user selections and user feedback in the user table but may link them and/or replicate them in a corresponding service provider in the service provider table.

In some embodiments, computing device 116 may analyze referral interactions to re-compute response times and referral completion metrics for determining available service providers. For example, computing device 116 may update response metrics associated with a service provider after each iteration of process 900 and/or following a subset of the process. For example, computing device 116 may update service provider information (e.g., in a service provider table) to identify service provider that on average respond within shorter time frames (e.g., within a threshold timeframe) or have a history of successful outcomes so that the machine learning model may assign them higher weights. Computing device 116 may also identify providers with patterns of delayed responses, repeated declines, or frequent non-responses to receive lower weights or be temporarily excluded from recommended results. In some embodiments, computing device 116 may anonymize and aggregate referral data of all users. Computing device 116 may use anonymized referral data to develop user analytics and service utilization forecasting. In some embodiments, computing device 116 may communicate service utilization forecasting to service providers and agencies (e.g., via service provider device 112f).

At step 915, computing device 116 renders the available service providers on the service platform. Computing device 116 may format each available service provider to be rendered as a selectable graphical element within a user interface of user device 112a-d. In some embodiments, the selectable graphical element may include a card, tile, or box. In some embodiments, the available service providers may be rendered as list. For example, the list may be ordered from most to least suitable for the user, as determined by the machine learning model in step 910. In other embodiments, computing device 116 may use colors, stars, and/or numbers. to identify the determined ranking of the available service providers. For example, computing device 116 may use five-star rating scores, numerical scores from 1-10, and/or color indicators ranging from green to red. In some embodiments, computing device 116 may render multiple lists of available service providers corresponding to different categories of service selected by the user.

At step 920, computing device 116 receives a user selection for a service provider from the rendered available service providers. Computing device 116 may receive the user selection through embedded coding on the service platform which associates each displayed service provider with a unique service provider identifier. When the user interacts with the selectable graphical element associated with a particular service provider, the embedded coding transmits the associated service provider identifier to computing device 116. For example, embedded coding on the service platform transmits the unique service provider identifier upon detecting a click, touch, or swipe. In some embodiments, computing device 116 may receive multiple selections from a user. For example, a user may desire to generate referral requests for multiple providers for a same service category or the user may select service providers across different selected service categories.

At step 925, computing device 116 retrieves data to generate a referral message that requests services from a user-selected service provider. In some embodiments, data to generate a referral message may include message components required to build the referral message according to a pre-stored template. For example, computing device 116 may generate and/or retrieve introductory text, the name of the service provider, a type of service being requested, instructions, and/or questions.

Computing device 116 may retrieve referral message data stored in one or more databases 118. In some embodiments, referral message data may be stored in the service provider tables located in one or more databases 118. Computing device 116 may use the service provider identifier retrieved at step 920 to search for and retrieve referral message data in the service provider tables. For example, computing device 116 may retrieve the name of the service provider, a person associated with the service provider, details on what user information should be retrieved, and/or how the referral message should be formatted (e.g., pre-stored template information).

In some embodiments, referral message data may be stored in the user tables located in the one or more databases 118 and/or may be retrieved from user information collected at step 905. Computing device 116 may use a user identifier (e.g., associated with an account) to search for and retrieve referral message data in the user tables. For example, computing device 116 may retrieve user information received by computing device 116 at step 905, including location, selected service category, data derived from the user device 112a-d, such as language preference and time zone, and information provided directly by the user, such as age range and living status.

Computing device 116 may use the user data to construct the referral message or may include the user data in the referral message. For example, computing device 116 may use the user data (e.g., data retrieved from the user table in database 118) to generate a summary of the user to include in the referral message. For example, the summary may recite “Hi, my name is John Smith. I'm looking for help with health. I live in zip code 12345. If you're able to help or guide me, I'd really appreciate it. Thank you.”

Computing device 116 may assemble the referral message using a pre-stored referral message template (e.g., stored in database 118), which includes predefined data fields that referral data can be organized into. For example, computing device 116 may insert retrieved service provider message data and/or user data into predefined fields.

Computing device 116 may attach metadata to the referral message that allows for tracking the referral message across communication channels. In some embodiments, computing device 116 may include the metadata as a parameter in a web link. In some embodiments, computing device 116 may include the metadata in a dedicated email header, within a body of an email, or as a parameter in a secure reply link contained in an email. In some embodiments, computing device 116 may include the metadata in a secure reply link contained in a SMS message. In some embodiments, computing device 116 may include the metadata as a field in a data payload of an API or webhook delivery. In some embodiments, computing device 116 may add the metadata as a hidden field linked to a message thread in an internal service platform interface. In some embodiments, the metadata may include a correlation identifier. The correlation identifier may be generated when a referral message is created and may include a time-based component, a random or pseudo-random sequence, a hashed combination of internal values, and/or routing identifiers. The attached metadata allows computing device 116 to track whether the user has received a response to the referral message, as further detailed below with respect to step 950.

At step 930, computing device 116 renders the referral message to be sent to the selected service provider. The referral message may be rendered on the user interface of the user device 112a-d. In some embodiments, computing device 116 may render the referral message in a text editable box and/or a partially text editable box where predefined portions of the referral message are fixed and user modifiable portions are selectively enabled to allow the user to add content. For example, the referral message may state an introductory statement, such as “A request has been submitted for assistance in the category of Housing Support. The client is currently located in the Riverside area and is seeking help related to unstable housing conditions.” The referral message may also state the user's relevant information, such as the user's name (e.g. “Name: Maria (preferred name provided by client)”), preferred language (e.g. “Preferred Language: English”), and/or location (e.g. “Location: 1.2 miles from your service area boundary”). The referral message may also include additional details about the client that computing device 116 received at step 905 (e.g., “The client indicated that she received an eviction notice and may need help identifying temporary housing options. She is available for follow-up by phone or email.”).

At step 935, computing device 116 receives user input regarding the referral message. Computing device 116 may enable the user to modify the generated referral message, add remarks, include supporting documentation, and/or send the referral message. Supporting documents may provide additional information relevant to the service being requested, for example, health insurance documentation.

In some embodiments, computing device 116 may include text prompts indicating to the user to add remarks and/or additional documentation to the referral message based on retrieved service provider requirements (e.g., retrieved from a service provider table). For example, a user may be prompted to add remarks indicating additional comments, contextual information, and/or concerns or upload documents supporting their request. Computing device 116 may present the text prompts as user interface elements on a user interface of the user device 112a-d. Computing device 116 may include the supporting documents as attachments to the referral message. In some embodiments, computing device 116 may store the remarks added or documents uploaded by the user in the user table. In some embodiments, adding at least one of remarks or additional documentation to the referral message may be optional, in which computing device 116 116 may transmit the referral message without additional content.

Computing device 116 may render a graphic element (e.g., a box, icon, label, button) to receive a user request to send the referral message to the service provider. For example, the user may request computing device 116 to send the referral message by tapping on a “send” button on the user interface of the user device 112a-d.

In some embodiments, steps 905-935 may occur over a first communication channel associated with the service platform. A first communication channel may include a service platform webpage or mobile application. The first communication channel may communicate information between computing device 116 and user device 112a-d.

At step 940, computing device 116 encrypts the referral message and sends the referral message to the selected service provider. Computing device 116 may encrypt the referral message data so that it is secured during transit and can only be decrypted by computing device 116 and the selected service provider. For example, before sending the referral message, computing device 116 may transform the text of the referral message into an unreadable format by scrambling the text of the message using a cryptographic key. A system of the selected service provider (e.g., 112f) may be equipped with a key that allows the system of the selected service provider to decrypt the referral message by restoring the referral message to a readable format. In some embodiments, the selected service provider may be equipped with the same key as computing device 116. In another embodiment, computing device 116 may use a cryptographic key that is different from the key that allows the selected service provider to decrypt the referral message, allowing only the selected service provider to decrypt the referral message. In the event that an unauthorized party without the correct key obtains the encrypted message, the data may remain encrypted.

Computing device 116 may send the referral message to the service provider over a second communication channel external to the service platform. Examples of second communication channels may include email, in-app messaging, web interface, SMS messaging, and/or API webhook. In some embodiments, computing device 116 may send the referral message over a type of second communication channel available to and/or preferred by the service provider. Computing device 116 may determine the available and/or preferred type of second communication channel using contact means data stored in the service provider table. For example, computing device 116 may use the service provider identifier to search the service provider table for contact means information (e.g., email, SMS message, etc.) associated with the service provider. Computing device 116 may also use the service provider identifier to search the service provider table for contact information of the user corresponding to the identified contact means. Computing device 116 may send the referral message to the identified contact over the identified type of second communication channel. In some embodiments, computing device 116 may send the referral message over more than one type of communication channel. In some embodiments, computing device 116 may simultaneously send more than one referral message. For example, computing device 116 may send more than one referral message if computing device receives multiple service category selections from the user.

At step 945, computing device 116 associates the user and the referral message in the one or more databases 118 with a timestamp indicating when the request was sent. Computing device 116 may store the referral message data and/or a timestamp in the user table. Computing device 116 may also store the associated correlation identifier in the user table. Computing device 116 may update a referral record stored in the user table with information about the referral message transmitted at step 940, including the timestamp and/or a date of the transmission. In some embodiments, computing device may update the user table and service provider table to link the information associated with the user stored in the user table with the service provider contacted at step 940 and the corresponding information stored in the service provider table.

In some embodiments, computing device 116 may encrypt data stored in the one or more databases 118. For example, computing device 116 may encrypt sensitive data stored in one or more databases 118, which may include personal identifiers, contact details, and referral content, using symmetric keys. The symmetric keys may consist of a singular secret key that is used for encrypting and decrypting the one or more databases 118. In some embodiments, certain fields in the database may require search capability, in which computing device 116 may use deterministic and/or format preserving encryption techniques to enable equality checks and/or index lookups without decrypting entire records. In some embodiments, deterministic methods may include computing device 116 applying the same encrypted value to a datum and format preserving methods may include computing device 116 keeping the encrypted data in the same format as the original data. In some embodiments, fields that do not require search capability may be encrypted with randomized techniques to maintain confidentiality. For example, computing device 116 may scramble readable text of confidential health information.

In some embodiments, computing device 116 may allow a user to access the user table using a verification credential associated with the correlation identifier. For example, computing device 116 may associate a verification credential with the correlation identifier attached to the referral message at step 925. The verification credential may include a token, password, signed link, user identification information such as a social security number and/or a numeric code. In some embodiments, the verification credential may be created when the user creates an authenticated account. In some embodiments, the verification credential may be created when the user first accesses the service platform. The verification credential may be stored in the user table. Computing device 116 may prompt a user attempting to access the user table to present the verification credential. In some embodiments, computing device 116 may present the prompt on a user interface of the user device 112a-d. Computing device 116 may authenticate the user by comparing the credential submitted by the user with the verification credential associated with the correlation identifier. Upon successful authentication, computing device 116 may allow access to information in the user table that is associated with the correlation identifier.

At step 950, computing device 116 tracks whether a response to the referral message was received. In some embodiments, the response may be received from the service provider through the second communication channel. In some embodiments, the service provider may respond through the same type of second communication channel that the referral message was sent through. For example, the service provider may respond to a referral request received via email by transmitting a reply message that is linked to the original electronic mail message thread.

Computing device 116 may track whether the response was received by tracking the metadata attached to the referral request. In some embodiments, computing device 116 may track whether a response was received using the correlation identifier, which may remain attached and/or may be automatically duplicated in the service provider response to the referral message. For example, computing device 116 may extract the correlation identifier from the response and use the correlation identifier to associate the response with the original referral message. Computing device 116 may associate the response with the original referral message if the correlation identifier extracted from the service provider response matches a correlation identifier embedded in the referral message. For example, upon extracting a correlation identifier of a response, computing device 116 may search for a corresponding correlation identifier in the user table. Computing device 116 may extract a timestamp indicating when the response was sent and store the response and the timestamp of the response with the user table

At step 955, computing device 116 monitors the time since the referral message was sent and sends reminders to the service provider. Computing device 116 may establish and store protocols for resolving unaddressed referral messages. For example, protocols may include one or more time thresholds, reminder message data, and/or rules for how computing device 116 responds to exceeding one or more time thresholds.

In some embodiments, computing device 116 may determine whether a time since sending the referral message has exceeded a first threshold. In some embodiments, computing device 116 may compute an elapsed time value at regular intervals by comparing the timestamp to a current system time. Computing device 116 may compare the elapsed time against a predetermined first threshold (e.g., two hours), which database 118 may store. Computing device 116 may generate and transmit a request reminder to the service provider in response to the elapsed time exceeding the first threshold. The request reminder may include a notification sent to the service provider consisting of a reference to the original referral, the correlation identifier, the transmission time, the elapsed time since submission of the referral message and/or a reminder message. In some embodiments, the notification may state that a referral submitted at an identified date and time, which may correspond to the date and timestamp stored in the referral record at step 945, has remained unanswered for longer than a determined response window (e.g., a first time threshold).

In some embodiments, sending a reminder request may include resending the initial referral request to the service provider. For example, a computing device 116 may update an email header or message body indicating this is the second time the referral request is being transmitted and include the contents of the first request reminder.

In some embodiments, computing device 116 may generate a new request for a second service provider if the time since sending the referral message exceeds a second threshold, which may be larger than the first threshold. For example, computing device 116 may select the service provider with the next highest rank (e.g., from a user table) and use the service provider identifier to search for and retrieve the corresponding referral message data from the service provider table. Computing device 116 may generate and transmit the new referral message as described above.

In some embodiments, computing device 116 may prompt the user to select another service provider if the time since sending the referral message exceeds the second threshold or a third threshold, larger than the second threshold. For example, computing device 116 may present a prompt in the service platform requesting the user to select another service provider from the list computing device 116 rendered to the user at step 915. In some embodiments, computing device 116 may repeat steps 920-955.

Computing device 116 may update the referral record associated with the user in the user table to include the reminder event and the corresponding data. The relevant data of the reminder event may include the time that the reminder was generated and the type of reminder that was issued. In some embodiments, sending a reminder request may prompt computing device 116 to display updated status information about the referral on the user interface of the user device 112a-d. For example, computing device 116 may present a message on the user interface that reads: “Awaiting provider response—reminder sent at 12:15 PM.”

At step 960, if a service provider response is received, computing device 116 transmits the service provider response to the user. Computing device 116 may extract text from the response and render the extracted text on the user interface of the user device 112a-d. For example, computing device 116 may extract text from the response and render it below the outgoing referral message on the same display,. Computing device 116 may transmit the service provider response to the user device 112a-d over the first communication channel.

In some embodiments, computing device 116 may execute steps 905-960 on the service platform.

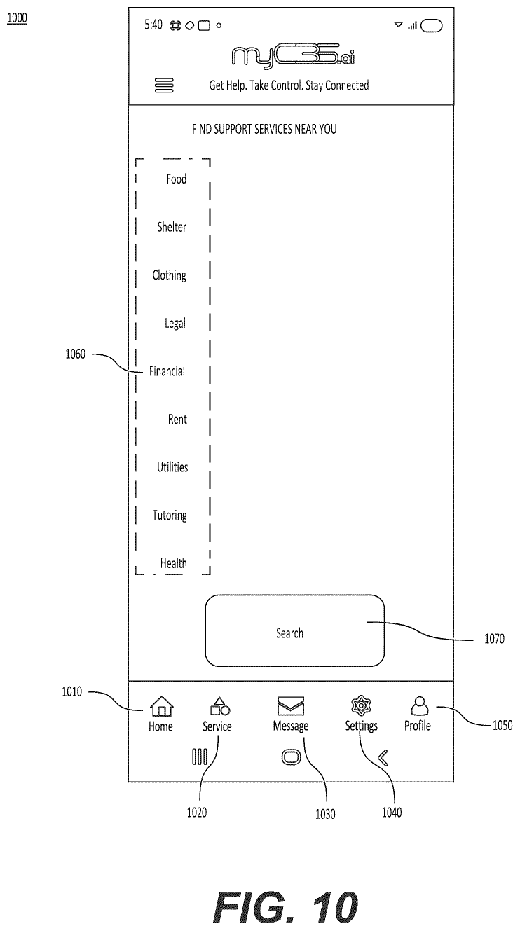

FIG. 10 illustrates an exemplary user interface display 1000 for an application configured to display a home screen and provide navigational access to various computing device 116 functions in accordance with embodiments of this disclosure. In accordance with embodiments, the user interface may correspond to the user interface of the user device 112a-d associated with the service platform previously disclosed in FIG. 1. In some embodiments, home icon 1010 may direct the user to the home screen display and service icon 1020 may direct the user to a display displaying available service categories. For example, a user tapping the service icon may cause the user interface to present a display of available service categories 1060, which may correspond to the service categories displayed in step 915 of FIG. 9.

The messages icon 1030 may direct the user to a display of a messaging inbox of the user containing messages sent to and received from one or more service providers. For example, a user tapping the messages icon may cause the user interface to present a display of referral messages and service provider responses, which may correspond to the display presented in steps 930 and 960 in FIG. 9.

Settings icon 1040 may direct the user to a display of configuration options for the application. The profile icon 1050 may direct the user to a display of access to the account and personal information of the user (e.g., information retrieved from database 118). Each icon may include a descriptive label below. In some embodiments, display 1000 may also include a touch-based search bar 1070, which enables a user to search for a service category or service provider using keyboard inputs when activated. For example, upon tapping the search bar 1070, available service providers may be determined, as described in step 915 above.

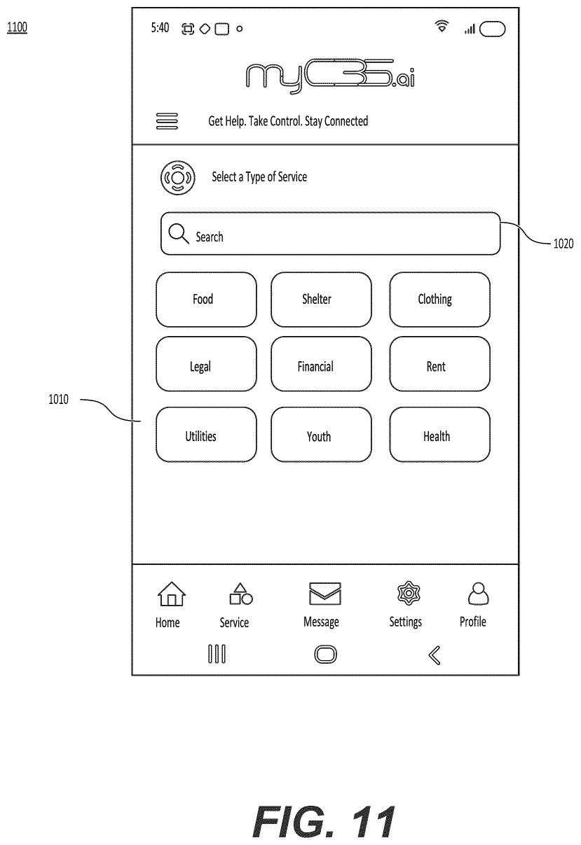

FIG. 11 illustrates an exemplary display 1100 for selecting a service category on the user interface of the user device 112a-d corresponding to step 905 in FIG. 9, in accordance with embodiments of the present disclosure. The user interface may correspond to the user interface of the user device 112a-d associated with the service platform previously disclosed in FIG. 1. Display 1100 may include a user interface element 1110 presenting the service categories available. In some embodiments, the user interface element may be a grid of tiles. In some embodiments, each tile may represent a service category. In some embodiments, display 1100 may also include a touch-based search bar 1120, which enables a user to search for a service category using keyboard inputs when activated.

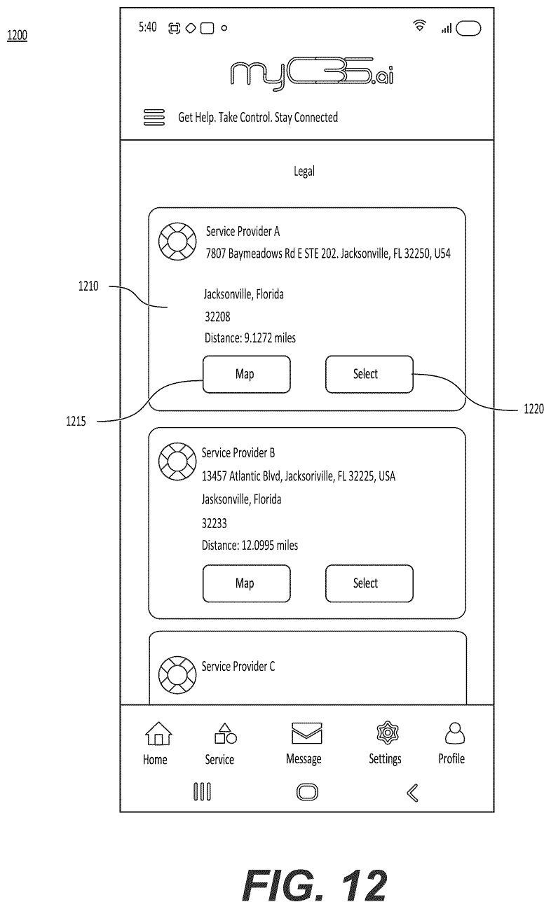

Selecting one of the service category tiles may direct the user to a display of a provider list that presents organizations matching the selected category and geographic filters on the user interface, as illustrated in display 1200 of FIG. 12. FIG. 12 may correspond to step 915 in FIG. 9, in accordance with the embodiments of the present disclosure. Display 1200 may present one or more display elements 1210 that may include provider information. In some embodiments the one or more elements may be cards, boxes, and/or tiles. Each display element 1210 may be an independently rendered container representing a unique service provider capable of offering assistance in the selected category. In accordance with embodiments, the service providers presented on this display may correspond to the available service providers determined at step 910 in FIG. 9.

In some embodiments, the one or more display elements 1210 may display a name of the service provider, an address of the service provider, and/or a distance between the location of the service provider and the location of the user device 112a-d. In some embodiments, display elements may also display availability, estimated response times, and other attributes retrieved from local cache or pre-computed analytics. The one or more display elements 1210 may also include a touch-responsive map icon 1215, which directs the user to a display of the location of the service provider on a map when selected. In some embodiments, the map icon may direct the user to an external navigation platform. The display element 1210 may also include a touch-responsive selection icon 1220, which indicates that the user has selected that particular service provider when selected. The selection icon 1220 may also initiate the next step in the workflow and direct the user to the corresponding display. In some embodiments, selection of the selection icon 1220 on a particular display element 1210 may direct the user to a display of a referral message preview, which may correspond to step 930 in FIG. 9, in accordance with the embodiments of the present disclosure.

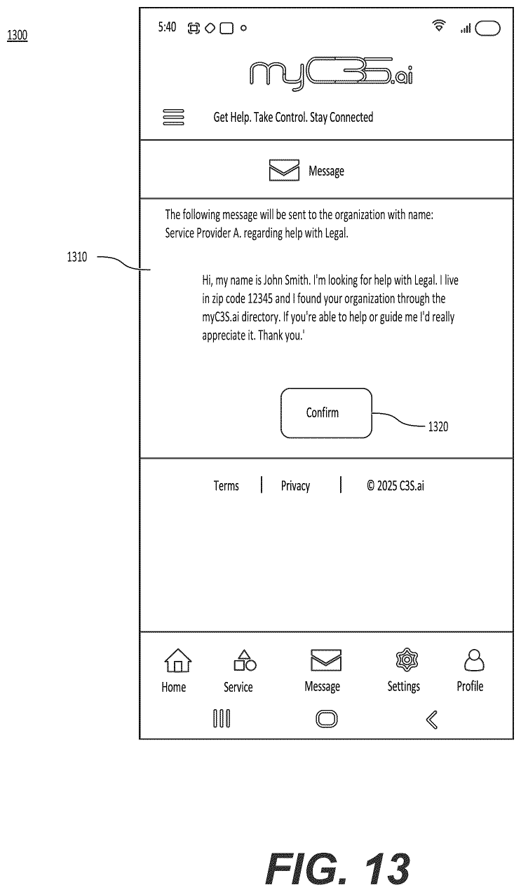

The referral message preview display 1300, as illustrated in FIG. 13, may be configured to present a preview and confirmation screen of the referral message that will be sent to the service provider, which may correspond to step 930 in FIG. 9, in accordance with the embodiments of the present disclosure. The user interface may correspond to the user interface of the user device 112a-d associated with the service platform previously disclosed in FIG. 1. Display 1300 may include a display element 1310 allowing the user to preview the referral message. The display element 1310 may identify the service provider the referral message is being sent to and the service category being requested (e.g. “The following message will be sent to the organization with name: Provider A, regarding help with Legal.”). The display element 1310 may also display the full text of the referral message to be sent to the service provider and a touch-responsive confirmation icon 1320, that may trigger computing device 116 to transmit the referral message to the service provider, in accordance with step 940 of FIG. 9, when selected by the user.

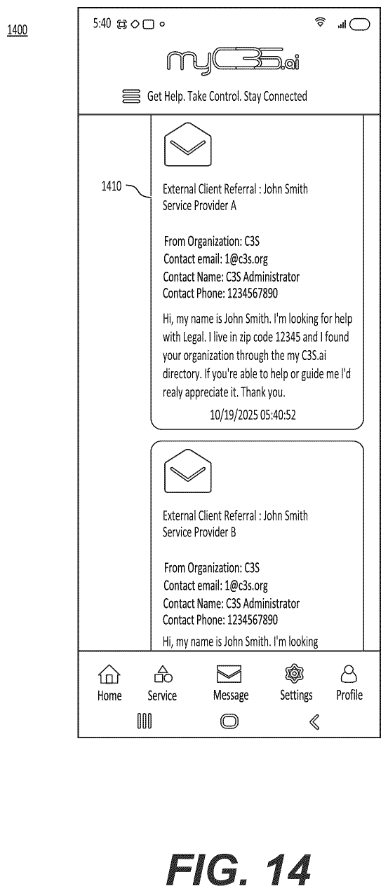

FIG. 14 illustrates an exemplary display 1400 of two-way messaging display on the user interface, which may correspond to step 960 in FIG. 9, in accordance with the embodiments of the present disclosure. The user interface may correspond to the user interface of the user device 112a-d associated with the service platform previously disclosed in FIG. 1. Display 1400 may present one or more display elements 1410. Each display element 1410 may be an independently rendered container. A display element 1410 may represent a message sent between the user and a service provider. A message may include a referral message sent to a service provider or a response to a referral message. In some embodiments, the display element 1410 may include a title or subject line identifying the type of referral, the name of the individual requesting service, and the name of the service provider the referral was sent to, the referral message generated by computing device 116, additional information relevant to the request, for example, a contact email, a contact phone number, or the name of the organization sending the referral message, and/or the date and time at which the referral message was sent. In some embodiments, unread messages may cause an alert icon to be displayed near the message icon.

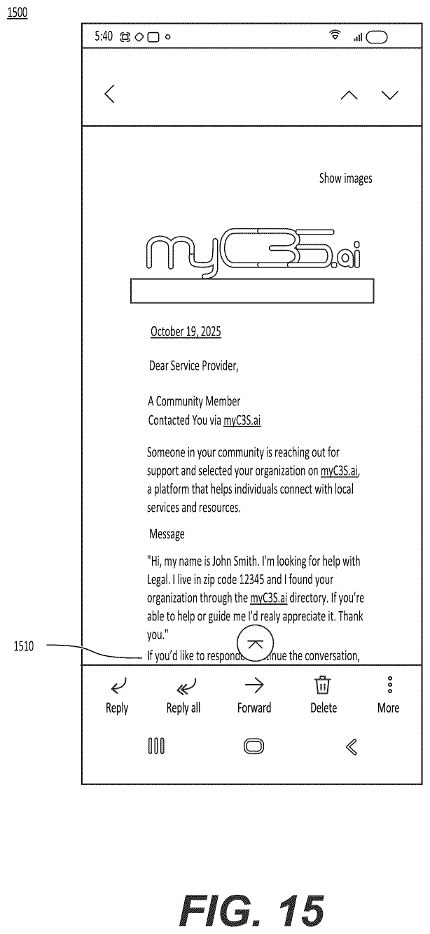

FIG. 15 illustrates an exemplary display 1500 of a notification received by a service provider, which may correspond to the referral message transmitted at step 940 in FIG. 9, in accordance with the embodiments of the present disclosure. The user interface may correspond to the user interface of the service provider device 112f disclosed in FIG. 1. Display 1500 may include a standard response statement 1510, which explains the next steps for the service provider in response receiving the referral message. The standard response statement may include a hyperlink that directs the service provider to a display that corresponds to the next step in the workflow. In some embodiments, the next step may be sending a response to the user, which may correspond to the response received at step 960 in FIG. 9, in accordance with the embodiments of the present disclosure.

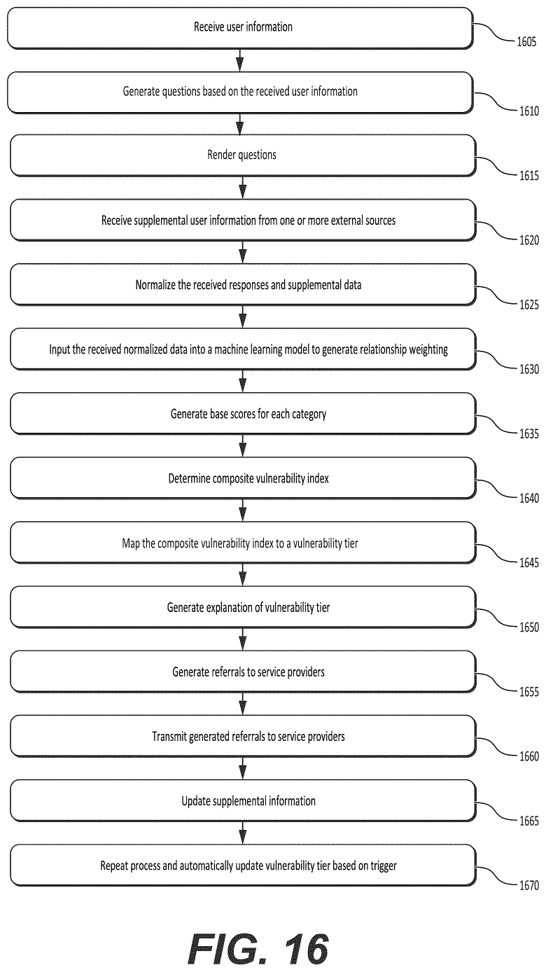

FIG. 16. describes an exemplary method 1600 of intelligent client intake and vulnerability tiering in accordance with embodiments of this disclosure.

At step 1605, computing device 116 receives user information. User information may comprise user-specific attributes that describe relevant characteristics of the user, including age, poverty level, demographics, and location. Service categories may include but are not limited to food, legal, housing, employment, healthcare, clothing, financial, youth, transportation, and education. In some embodiments, computing device 116 may prompt the user to enter user information and/or may retrieve user information (e.g., from a user table, as described above). In some embodiments, computing device 116 may retrieve user location, a selected service category, and/or user eligibility information as described in step 905 of FIG. 9. Computing device 116 may store the user information in the user table and associate the user information with the user. In some embodiments, computing device 116 may receive an identifier associated with a service organization or agency through whom a user is answering the questionnaire.

At step 1610, computing device 116 generates questions to present based on the received user information. Questions may be presented in the form of the questionnaire. Computing device 116 may generate questions that cover service categories including housing, food security, safety, mental health, youth issues, and/or life support.

In some embodiments, computing device 116 may generate questions using a schema-agnostic questionnaire compiler. The questionnaire compiler may include a machine learning model configured to evaluate the user information retrieved at step 1605 to determine an appropriate question set to render. In some embodiments, the questionnaire compiler may select questions from one or more question groups identified by service providers. For example, in some embodiments, a questionnaire may be developed for a specific service provider through whom the user is answering the questions.

The question groups may comprise questions relevant to evaluating a user's alignment with the services offered by a particular service provider and may be tailored to different user characteristics, such as age ranges, income levels, demographic profiles, or geographic regions. Database 118 may store multiple question groups. The machine learning model may reference the user information and evaluate whether question groups align with the user's attributes (e.g., user information retrieved in step 1610). In some embodiments, the machine learning model may apply weights to the user information to determine which of the corresponding question groups carry the strongest correlation to the user. The questionnaire compiler may use the output of the machine learning model to select and assemble a question set that is tailored to the user's characteristics. For example, the questionnaire compiler may apply a relatively high weight to the age of the user. Based on the weighting, the questionnaire compiler may select a question group comprising questions associated with the user's age or age range.

In some instances, computing device 116 may retrieve from database 118 question branching logic inputted from service providers, which consists of branching logic, which dynamically adapt based on the previous responses of the responder. Computing device 116 may generate questions to ensure compliance with conditions, requirements, and eligibility procedures of service providers (e.g., branching logic), and local jurisdictions. For example, computing device 116 may incorporate these requirements into its machine learning model to select questions that align with the user while also satisfying particular jurisdictional and service provider requirements.

Computing device 116 may generate questions that allow for multiple answer selections or have multiple parts. Computing device 116 may generate questions in a user-selected language and in an accessibility-compliant format. Computing device 116 may dynamically expand or collapse questions based on previous responses (e.g., by referring to stored logic from service providers). For example, if a user indicates that their housing situation is unstable, computing device 116 may generate additional questions related to the type of instability, including whether the user has received an eviction notice, is staying temporarily with others, or does not have a regular place to stay. If the user selects that their housing is stable, computing device 116 may not present these additional questions.

At step 1615, computing device 116 renders the questions generated by computing device 116 at step 1610 to the user. The questions may be transmitted over network 114 and rendered in the platform on the user interface of the user device 112a-d. Computing device 116 may receive responses to the questions over network 114 and store them one or more databases 118.

At step 1620, computing device 116 receives supplemental user information from one or more external sources. Computing device 116 may receive the supplemental data from one or more integrated data sources. For example, integrated data sources may include electronic health record (EHR) systems and statewide automated child welfare information systems (SACWIS). EHR data may include encounter history, diagnoses, or emergency-departure utilization. SACWIS data may include case status, placement history, or service interactions. In some embodiments, computing device 116 may receive supplemental data through an integration layer configured to securely exchange client data with external systems via standardized protocols and interfaces (e.g., HL7 FHIR and REST APIs). The integration layer may be configured to comply with data privacy rules and data-sharing agreements of service providers and local jurisdictions. The integration layer may be encrypted and maintained under access-based control permissions. Accordingly, the integration layer may be compliant with data privacy laws and regulations.

In some embodiments, when the user begins the intake process at step 1605, computing device 116 may send a request to the relevant integrated data source. The request may be sent using a secure application programming interface. The request may contain one or more authorized identifiers, which may include a user-specific token, case number, or an alternative reference value that is used to identify information in third-party records and permitted by the agency's data-privacy policy. The external system of the data source may process the request and return a structured response containing the information approved for distribution by the agency. Computing device 116 may convert the information received from the external system into a standard internal format and associate the received information with the current intake session of the user. Computing device 116 may encrypt and/or store the information received from the external system and one or more authorized identifiers in the user table.

At step 1625, computing device 116 normalizes the received user responses and supplemental data. Normalization may consist of computing device 116 converting user responses and supplemental data into a consistent format to allow for uniform processing of information. In some embodiments, computing device 116 may normalize user responses, third-party record information, and service provider information, including specialized terms and identified question groups, by mapping them to canonical need ontologies. For example, computing device 116 may determine the meaning of one or more user responses, third party or service provider specialized terms, and/or identified questions and align them with pre-stored internal terminology.

In some embodiments, normalization may include mapping expressions or labels of user responses and questions identified by service providers to a single internal category or terminology. For example, service provider A may provide a question describing whether an eviction notice was received, while service provider B may provide a general housing stability indicator. Computing device 116 may map these question-based points of data to a common internal category. Normalization may also consist of computing device 116 standardizing a format for data including dates, timestamps, income inputs, or location information. In some embodiments, if computing device retrieves information from outside sources, computing device 116 may extract the information relevant to the intake or referral process and match the information to the corresponding internal category. Computing device 116 may also store the information in one of the databases 118, for example, the third-party records table.

At step 1630, computing device 116 inputs the normalized data and user information into a machine learning model to generate relationship weighting for the user responses. A weight for a user response may indicate a level of significance of the user response in evaluating a situation of a user. In some embodiments, level of significance may correspond to a strength of association between the user response and one or more other user responses, service-provider requirements, and/or pre-stored criteria.

In some embodiments, the machine learning model may be trained (e.g., by computing device 116) to assign weights to the user responses using a supervised learning algorithm trained on historical data. Historical data may include service provider records and prior referral outcomes. In some embodiments, historical data may also include prior machine learning model outputs. Inputting the historical data into the machine learning model may enable the model to learn patterns and relationships among user responses by correlating combinations of user responses with prior service provider records, referral outcomes, and prior model outputs, teaching the model how different user responses interact to influence user needs and service outcomes. To identify correlations, the machine learning model may be trained to extract associations and determine a relationship between at least two of the user responses using the algorithms disclosed above. Supervised learning algorithms may include Naïve Bayes, K-Nearest Neighbors (KNN), Calculation and Regression Tree (CART), Linear Regression, Logistic regression, or other regression algorithms that may be trained to assign weights based on evaluations of data.

In some embodiments, service providers may define domain weights applied to user responses that may reflect organizational priorities, policy objectives, or program-specific needs. Domain weights may be assigned to individual questions or categories of questions and stored in one or more databases 118. For example, a service provider operating a housing-focused program may assign higher weights to questions related to eviction risk, unsafe living conditions, or loss of housing, while a youth services service provider may assign higher weights to questions related to school attendance, personal safety, or caregiver supervision. In some embodiments, the assigned weights remain fixed with respect to the organization and are applied uniformly regardless of a particular user's responses. In some embodiments, one or more algorithms may evaluate historical outcomes, referral effectiveness, or aggregate scoring distributions to assess the validity of the domain weights determined by the service providers and may generate recommended adjustments to the domain weights. The domain weights may also be predetermined by the location of the user device 112a-d. For example, domain weights for a user in a rural southern-region town may be predetermined and different from domain weights for a user in an urban northeastern-region city due to historically different needs, resources, and types of service providers. The domain weights may be recalibrated to ensure demographic bias is excluded from vulnerability index results. In some embodiments, the domain weights may be recalibrated using statistical validation techniques, fairness metrics, and/or outcome-based analysis.

Computing device 116, via the machine learning model, may assign user response weights based on correlations identified between user responses, service provider requirements, and/or pre-stored criteria. In some embodiments, computing device 116 may assign higher weights to user responses with high correlation to critical conditions. For example, user responses indicating housing instability, child safety, and urgent health concerns may receive a higher weight be and considered more relevant than user responses indicating transportation. In some embodiments, computing device 116 may assign higher weights to user responses with high correlation to user responses in different categories. For example, a user response indicating a feeling of depression in a mental health category may be given more weight based on a user response indicating no stable housing in a housing category. Similarly, a user response related to household safety may receive a higher weighting if another user response indicates that a child is present in the home. In some embodiments, computing device 116 may assign user response weights based on correlations to regional and service provider policies, and user location information. For example, user responses indicating a need for transportation services may be weighted higher if the location information of the user indicates an isolated geographic region. In some embodiments, the machine learning model may use location information of the user to determine weight assignments.

At step 1635, computing device 116 generates base scores for each service category. Computing device 116 may generate base scores by combining the weighted user response values determined by computing device 116 at step 1630 for each category and adjusting resulting values of the combination. Computing device 116 may use a hybrid scoring system to adjust the values produced from combining the weighted user responses. The hybrid scoring system may include rule-based logic and machine-learned modifiers. Computing device 116 may use the hybrid scoring system to apply at least one of rule-based logic and machine-learned modifiers to the weights of the user responses and/or base scores. In some embodiments, the hybrid scoring system may apply a combination of rule-based logic and the machine-learned modifiers to the weight of a service category to compute the base score of the service category. For example, the hybrid scoring system may apply rule-based logic to the weight of a user response to produce an initial adjustment and may subsequently apply machine-learned modifiers to further adjust the weight to compute a base score for a service category. In some embodiments, computing device 116 may implement the hybrid scoring system in a federated learning architecture. A federated learning architecture may enable multiple entities to train a shared, decentralized model without exchanging raw data associated with the individual entities. In the present disclosure, computing device 116 may coordinate multiple hybrid scoring systems to train a shared model that is external to each hybrid scoring system, without requiring the hybrid scoring systems to share raw data, such as user information.

Rule-based logic may comprise one or more predetermined scoring rules used by computing device 116 to enforce restraints on the weights of the user responses and/or base scores. In some embodiments, rules of the rule-based logic may be defined by service providers. For example, the rules may correlate to service provider requirements. Rules defined by service providers may be stored in the service provider table and computing device 116 may use a service provider identifier to retrieve rules defined by a specific service provider. Computing device 116 may use the rules to set minimum score values, fixed user response weights, and conditional score adjustments. For example, if a calculated category score based on weighted user responses is below a predefined minimum value of 0.05, computing device 116 may elevate the category score to 0.05. In another example, if a service provider indicates that a user response to a question provided by the service provider should remain fixed, computing device 116 may prevent the question weight from being changed by the machine-learned modifiers. In another example, if the user responses include two predetermined user responses, 116 may require a corresponding category score to increase by an additional 0.1 beyond the value initially computed.

Computing device 116 may use machine-learned modifiers to adjust weights of the user responses and/or base scores using patterns learned from prior intake and outcome data. Prior intake data may include user information collected by computing device 116 at step 1605. In some embodiments, computing device 116 may use user information collected from one or more users within system 100. In some embodiments, prior outcome data may include service provider review and analytical information. In some embodiments, computing device 116 may retrieve prior intake and outcome data from the user table and the service provider table. In some embodiments, machine-learned modifiers may refine the weights of user responses and/or base scores results from the rule-based logic. Computing device 116 may use machine-learned modifiers to apply bonuses, overrides, and floor constraints to values. Computing device 116 may apply bonuses to increase the values of weighted user responses and/or category base scores when corresponding factors have demonstrated strong relevance. Computing device 116 may apply overrides to selectively alter portions of a rule-based score when contextual exceptions are detected. Computing device 116 may apply floor constraints to prevent scores from falling below predefined minimum levels when specific risk indicators are present.

At step 1640, computing device 116 determines a composite vulnerability index. The vulnerability index may be a predictive indicator of the urgency of an individual's service needs across one or more service categories. Computing device 116 may compute the composite vulnerability index by detecting and quantifying compounding vulnerabilities across multiple categories and aggregating the base scores determined at step 1635. Vulnerabilities may be risk indicators associated with the user that correspond to needs within one or more service categories. Computing device 116 may aggregate the base scores determined at step 1635 by combining the base scores into a single composite value. In some embodiments, computing device 116 may apply domain weights to the base scores to compute domain scores for each service category, in which computing device 116 may aggregate the domain scores into the single composite value. The domain weights may be defined by service providers or predetermined by geographic configuration, such as the location of the user device 112a-d. Computing device may modify the single composite value using vulnerability scores to form the composite vulnerability index.

Computing device 116 may detect and quantify compounding vulnerabilities across multiple categories from categorized user responses. Detecting compounding vulnerabilities may include identifying conditions where risk factors associated with different service categories interact in a way that impacts the overall vulnerability of the user significantly higher than the sum of risk factors individually. In some embodiments, computing device 116 may identify risk factors from the user's responses received at step 1615. For example, housing instability and unemployment may each represent separate risk factors, but when they occur together, the combined impact on overall vulnerability is significantly higher than the simple sum of the two. If the responses of the user indicate an existence of both, computing device 116 may assign a compounded risk value that reflects the amplified effect of the combination on the user's overall vulnerability.

In some embodiments, computing device 116 may use an automated combination function to quantify compounding risk, which is an algorithmic method that merges inputs from multiple categories to determine compounded risk. The combination function may employ one or more mathematical or algorithmic functions, including weighted multipliers, conditional modifiers, or non-linear scoring rules, to determine scores for compounding vulnerabilities. The combination function may use weighted multipliers to apply a scaling factor to service categories based on the presence or magnitude of other categories, proportionally increasing or decreasing the contribution of that category to overall vulnerability. The combination function may use conditional modifiers to amplify and/or suppress vulnerability scores when predefined conditions are satisfied. The combination function may use non-linear scoring rules to apply mathematical functions where changes in vulnerability scores are not directly proportional to input values. For example, the combination function may apply an exponential increase in vulnerability scores when multiple vulnerabilities are present. Computing device 116 may use the vulnerability scores to modify the single composite value to produce the composite vulnerability index.

In some embodiments, if any service category has a base score above a predetermined critical threshold, computing device 116 may assign a maximum value to the vulnerability index. In some embodiments, if two or more categories exceed a high-risk score threshold, computing device 116 may assign a maximum value to the vulnerability index. The vulnerability index may range from 0.1 to 1, with 0.1 indicating a lowest vulnerability and 1 indicating a highest vulnerability. In some embodiments, computing device 116 may store the vulnerability index in the user table.

At step 1645, computing device 116 maps the composite vulnerability index to a vulnerability tier. Computing device 116 may compare the vulnerability index score to vulnerability index score thresholds associated with different vulnerability tiers to determine a vulnerability tier for the user (e.g., Tier 1 to Tier 5). In some embodiments, computing device 116 may store the vulnerability tier in the user table.

In accordance with some embodiments, computing device 116 may automatically update thresholds used in steps 1640-1645 based on an initiating event. The initiating event may include new supplemental user information, service provider data, intake information, health data, welfare case data, and outcome data. For example, computing device 116 may automatically reevaluate and redefine vulnerability index score thresholds if computing device 116 receives agency data indicating that certain groups are not being accurately represented based on data from agencies may automatically cause. In another example, an update in the service policy of a service provider may automatically cause computing device 116 to reevaluate and redefine vulnerability index score thresholds.

At step 1650, computing device 116 generates a vulnerability tier record, which may include information associated with the vulnerability tier of the user. The vulnerability tier record may include an explanation of the vulnerability tier. The vulnerability tier record and/or explanation of the vulnerability tier may be presented to one or more service providers (e.g., via service provider device 112f). The explanation may include the vulnerability tier assigned to the user and identification of critical user responses and external factors impacting the vulnerability tier assigned to the user. Critical user responses may correspond to user responses receiving the highest weights in step 1630. Critical user responses may also be defined as user responses related to the service category having the highest base score. The explanation of the vulnerability tier may also provide an explanation of how social, health, and welfare factors collectively influenced the vulnerability tier of the user.

In some embodiments, the explanation may also present fairness diagnostics to authorized service providers. The fairness diagnostics may evaluate whether the process of assigning the vulnerability tier occurred in an equitable and unbiased manner across different groups or conditions. In some embodiments, the explanation may be used to generate a case summary document. The explanation may also include interactive visualizations of factor weights and historical comparisons. Interactive visualizations may include a bar chart illustrating the weights assigned to each question user response and service category. In some embodiments, the bar chart may include sliders allowing service providers to manually manipulate weights to see how they impact the vulnerability index value and vulnerability tier. In alternative embodiments, the bar chart may be touch enabled to present a description of the weighted factor when a bar is selected by a service provider. Interactive visualizations may also include a line chart or layered graph that compares historical trends of weight assignments for user responses and service categories. In some embodiments the explanation of the vulnerability tier may include a case summary of the user. The case summary may include a comprehensive description of information relating to the vulnerability tier of the user, including compounding vulnerabilities (e.g., “The client demonstrates compounding vulnerabilities across housing stability, safety, and healthcare access.”), key contributing factors (e.g., “Key contributing factors include an imminent risk of eviction, safety concerns within the household, and limited engagement with primary care.”), and recommended action (e.g., “Recommended action: Immediate referral to emergency housing support and safety planning. Human review required prior to approval.”). Computing device 116 may automatically generate the case summary upon identifying a vulnerability tier at step 1645. In some embodiments, computing device 116 may present the case summary as a document for review and approval by a human. The vulnerability tier record and/or explanation of the vulnerability tier may be presented to service providers on a graphical user interface (e.g., a dashboard) integrated with the service provider device 112f. In some embodiments, computing device 116 may store the explanation of the vulnerability tier in the user table.

At step 1655, computing device 116 generates referrals to service providers. Generating referrals may include the process described at step 910 in FIG. 9. Computing device 116 may generate referrals to service providers associated with the vulnerability tier assigned to the user. Association may be based on provider-specific attributes and trends including availability window, frequency and rate at which requests are accepted and fulfilled, average response time, and success rates of past referrals. Computing device 116 may use the service provider identifier to retrieve service provider-specific trends and attributes from the service provider table. For example, computing device 116 may generate referrals only to service providers with a 100 percent fulfilled request rate if the user is assigned a vulnerability tier of 5. In some embodiments, computing device 116 may use the vulnerability tier of the user to assign thresholds for the amount of time that elapses before computing device 116 sends a reminder request or undergoes an escalation action. The escalation action may include prompting the user to select another service provider to send the referral message to or automatically rerouting the referral message to an alternative service provider.

At step 1660, computing device 116 transmits generated referrals to service providers associated with the identified needs of the user. Computing device 116 may also track referrals after referrals are transmitted. Transmitting and tracking referrals may include the process described in steps 940-960 in FIG. 9. Computing device 116 may establish protocols for resolving unaddressed referral messages. Protocols may be based on the vulnerability tier of the user.

At step 1665, computing device 116 updates the user information of the user. For example, computing device 116 may update a user table to include responses, base scores, a vulnerability tier, service provider communications and metadata, and/or any other information collected during process 1600. Computing device 116 may also store the vulnerability tier and the explanation within the EHR and SACWIS records of the client.

At step 1670, computing device 116 repeats the process and updates the vulnerability tier in response to a trigger. Repeating the process may include repeating at least one of steps 1605-1665. Computing device 116 may initiate repeating the process at any point of steps 1605-1665. The trigger may include updated supplemental information, service provider data, intake information, health data, welfare case data, external source data, and outcome data. For example, changes to the EHR or SACWIS data of the user may cause computing device 116 to automatically generate questions to render to the user, in accordance with step 1610. In another example, the machine learning model may adjust user response weights to adhere to changes in regional and service provider policies, in accordance with step 1630. Computing device 116 may automatically check integrated third-party systems for updates at a predetermined frequency. In some embodiments, the frequency is daily. Computing device 116 may check for differences between current intake information and the previous intake information when a new intake is completed. The trigger may also include the arrival of a scheduled update or an amount of time since a previous update surpassing a predetermined threshold. For example, computing device 116 may automatically render a new set of questions to the user it has been 365 days since the last repeat of the process.

In an alternative embodiment, the user may use the service platform on the user device 112a-d to send a referral message with computing device 116 to at least one of the plurality of service providers 110c. In some embodiments, the at least one of the plurality of service providers 110c may provide a response to the referral message. In alternative embodiments, the at least one of the plurality of service providers 110c may forward the referral message to other service providers. A response from a service provider may be embedded with the same code. Upon receiving a response to the referral message, computing device 116 may match the code of the response to the code of the referral message and present referral messages and responses on one interface. The interface may be displayed on the user device 112a-d. In some embodiments, service providers may not need to be included in the provider network disclosed in FIG. 6 to receive and respond to referral messages. Computing device 116 may assign users a vulnerability tier, which may correspond to steps 1605-1670 in FIG. 16. Computing device 116 may use the vulnerability tier to determine a service provider to send the referral to, a response time, a frequency of follow ups, and/or conditions for escalation or rerouting actions. For example, if computing device 116 assigns a user a vulnerability tier of 5, computing device 116 may choose to re-route the referral message after a 30-minute period. In some embodiments, computing device 116 may use a combined rule system to manage referral message rules across more than one platform. For example, computing device 116 may control overrides, reminders and escalation actions for referral messages. Computing device 116 may also be configured to send one or more referral messages to one or more service providers at the same time. For example, computing device may send an individual referral message for housing, food, and counseling at the same time if the user indicates needs in all three service categories. Computing device 116 may create a case record that is associated with the user. Computing device 116 may store referral activity, referral messages, service provider replies, and the vulnerability tier of the user in the case record. In some embodiments, the case record may be stored on the user device 112a-d.

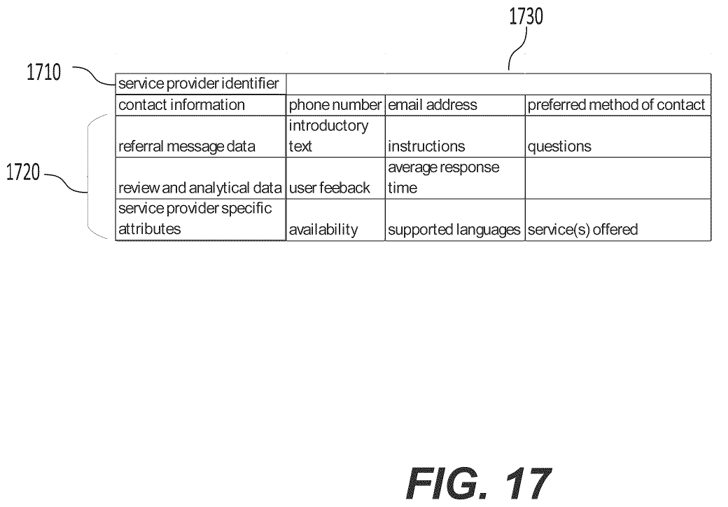

FIG. 17 illustrates an exemplary service provider table in accordance with embodiments of this disclosure. Computing device 116 may use the service provider table 1700 to store and/or retrieve information and/or data associated with a service provider. The service provider table may include the service provider identifier 1710, which computing device 116 may use to navigate the service provider table. The service provider table 1700 may include different types 1720 of service provider associated information, such as contact information, referral message data, review and analytical data, and service provider attributes. Within the different types of information associated with a service provider are indicators 1730 for specific types of data or information. For example, the referral message data associated with a service provider may include introductory text, instruction, and questions.

FIG. 18 illustrates an exemplary user table in accordance with embodiments of this disclosure. Computing device 116 may use the user table 1800 to store and or retrieve information and/or data associated with a user. The user table may include the user identifier 1810, which computing device 116 may use to navigate the user table. The user table 1800 may include different types 1820 of user associated information, such as referral record, system rules user information, correlation identifiers, vulnerability data, documentation, and contact information. Within the different types of information associated with a user are indicators 1830 for specific types of data or information. For example, the vulnerability information associated with a user may include a vulnerability tier and a vulnerability explanation.

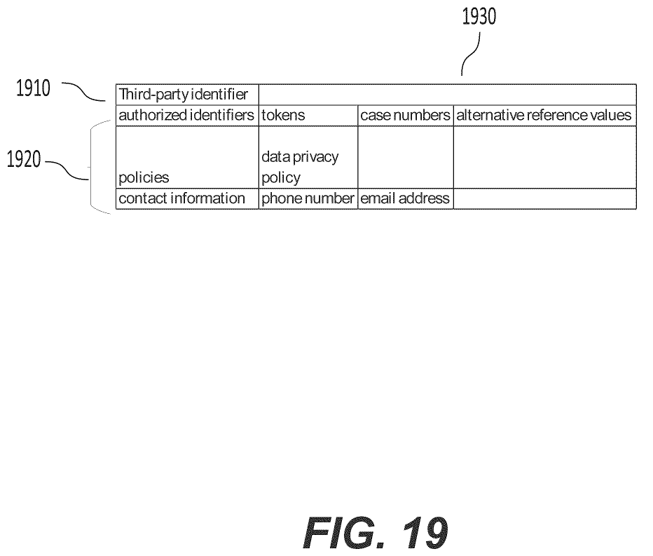

FIG. 19 illustrates an exemplary third-party records table in accordance with embodiments of this disclosure. Computing device 116 may use the third-party records table 1800 to store and or retrieve information and/or data associated with a third-party, such as an agency. The third-party records table may include the third-party identifier 1910, which computing device 116 may use to navigate the third-party table. The third-party table 1800 may include different types 1820 of third-party associated information, such as authorized identifiers, policies, and contact information. Within the different types of information associated with a third-party are indicators 1830 for specific types of data or information. For example, the contact information associated with a third-party may include a phone number and email address.

Although system 100 is described as being used to determine user health and behavioral needs, as explained previously, this is only exemplary. In general, the systems and methods of the current disclosure may be used to assess any of a variety of needs predicated on the suer's socio-economic status, health, behavior, environment, etc. While illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

1. A system for assessing user need based on relationship association and vulnerability tiering, the system comprising:
at least one processor; and
a storage medium storing instructions that, when executed, configure the at least one processor to perform operations comprising:
transmitting to a user device for display on a user interface, over a communication network, a questionnaire comprising questions that represent a plurality of categories;
receiving responses to the questionnaire from the user device over the communication network;
retrieving supplemental user information from one or more data sources;
converting the responses and the supplemental user information;
assigning, using a machine learning model, weights to the responses based on correlations among the responses;
computing base scores for the plurality of categories from the weighted responses using a hybrid scoring system;
quantifying, using a combination function, compounding vulnerabilities across categories;
applying the compounding vulnerabilities to the base scores to produce a vulnerability index score;
comparing the vulnerability index score to thresholds associated with vulnerability tiers to establish a vulnerability tier of a user; and
generating and transmitting a vulnerability tier record to the user on a graphical user interface, including an explanation of the vulnerability tier of the user.

2. The system of claim 1, wherein the operations further comprise recalculating the vulnerability tier of the user automatically in response to detecting an initiating event.

3. The system of claim 2, wherein the initiating event includes at least one of: changes to at least one of the supplemental user information from the one or more data sources or changes to the responses to the questionnaire.

4. The system of claim 1, wherein the operations further comprise:
selecting a service provider based on the vulnerability tier of the user;
generating a referral message; and
transmitting the referral message to the service provider.

5. The system of claim 4, wherein the operations further comprise:
monitoring an elapsed time after transmitting the referral message to the service provider; and
transmitting at least one of a reminder to the service provider or an alternative referral message to at least one alternative provider when a predetermined threshold for an elapsed time without receiving a response from the service provider is met,
wherein the predetermined threshold is based on the vulnerability tier of the user.

6. The system of claim 1, wherein the operations further comprise: dynamically rendering questions of the questionnaire based on previous responses of the user.

7. The system of claim 1, wherein the operations further comprise: identifying a user as a highest vulnerability tier upon determining one category has a base score above a threshold.

8. The system of claim 1, wherein the one or more data sources includes an electronic health record (EHR) system or a statewide automated child welfare information system (SACWIS).

9. The system of claim 1, wherein the hybrid scoring system includes rule-based logic and machine-learned modifiers,
wherein the machine-learned modifiers include bonuses, overrides, or floor constraints to compute base scores.

10. The system of claim 1, wherein the hybrid scoring system includes rule-based logic and machine-learned modifiers, and the rule-based logic is retrieved based on a service provider identifier associated with a service provider.

11. The system of claim 1, wherein the explanation of the vulnerability tier of the user includes identifying responses and external factors affecting the vulnerability tier of the user.

12. The system of claim 1, wherein transmitting the explanation of the vulnerability tier of the user includes rendering interactive visualizations on a user device.

13. The system of claim 1, wherein the operations further comprise: utilizing service provider outcome data to refine the weights and vulnerability tier thresholds.

14. The system of claim 1, wherein the operations further comprise: retrieving referral message data to be transmitted electronically to a service provider system based on the vulnerability tier of the user.

15. The system of claim 1, wherein the operations further comprise storing the vulnerability tier of the user and explanation of the vulnerability tier of the user within both EHR and SACWIS records of the user.

16. The system of claim 1, wherein the operations further comprise retrieving one or more of the questions of the questionnaire based on a service provider identifier.

17. The system of claim 1, wherein the system is implemented on a mobile application or a web-based application.

18. The system of claim 1, wherein at least one of the weights assigned to the responses is retrieved based on a service provider identifier.

19. The system of claim 1, wherein converting the responses and the supplemental user information includes mapping provider-specific intake terms to canonical need ontologies.

20. The system of claim 1, wherein the hybrid scoring system is implemented in a federated-learning architecture.

21. The system of claim 1, wherein the explanation of the vulnerability tier of the user includes a case summary of the user,
wherein the case summary is automatically generated and requires human review and approval.

22. A method for assessing user need based on relationship association and vulnerability tiering, comprising:
transmitting to a user device for display on a user interface, over a communication network, a questionnaire comprising questions that represent a plurality of categories;
receiving responses to the questionnaire from the user device over the communication network;
retrieving supplemental user information from one or more data sources;
converting the responses and the supplemental user information;
assigning, using a machine learning model, weights to the responses based on correlations among the responses;
computing base scores for the plurality of categories from the weighted responses using a hybrid system;
quantifying, using a combination function, compounding vulnerabilities across categories;
applying predetermined domain weights to the base scores to produce a vulnerability index score;
comparing the vulnerability index score to thresholds associated with vulnerability tiers to determine a vulnerability tier of a user; and
generating and transmitting a vulnerability tier record to the user on a graphical user interface, including an explanation of the vulnerability tier of the user.

23. A non-transitory computer-readable medium storing a set of instructions that, when executed by one or more processors, cause the one or more processors to perform operations comprising:
transmitting to a user device for display on a user interface, over a communication network, a questionnaire comprising questions that represent a plurality of categories;
receiving responses to the questionnaire from the user device over the communication network;
retrieving supplemental user information from one or more data sources;
converting the responses and the supplemental user information;
assigning, using a machine learning model, weights to the responses based on correlations among the responses;
computing base scores for the plurality of categories from the weighted responses using a hybrid system;
quantifying, using a combination function, compounding vulnerabilities across categories;
applying predetermined domain weights to the base scores to produce a vulnerability index score;
comparing the vulnerability index score to thresholds associated with vulnerability tiers to determine a vulnerability tier of a user; and
generating and transmitting a vulnerability tier record to the user on a graphical user interface, including an explanation of the vulnerability tier of the user.
